(19) United States
(12) Patent Application Publication
(10) Pub. No.: US 2026/0265691 A1
(43) Pub. Date: Sep. 10, 2026

(54) METHODS TO IMPROVE THE PERSISTENCE OF DOUBLE NEGATIVE T CELLS

(71) Applicant: University Health Network, Toronto (CA)

(72) Inventors: Li Zhang, Toronto (CA); Hyeonjeong Kang, Vaughan (CA); Jongbok Lee, Toronto (CA)

(21) Appl. No.: 18/876,486

(22) PCT Filed: Jun. 30, 2023
(86) PCT No.: PCT/CA2023/050899

§ 371 Date: Dec. 18, 2024

Related U.S. Application Data
(60) Provisional application No. 63/357,120, filed on Jun. 30, 2022.

(51) Int. Cl. C12N 5/0783; A61K 40/11; A61K 40/31; A61K 40/42; A61P 35/00

(52) U.S. Cl. CPC C12N 5/0636; A61K 40/11; A61K 40/31; A61K 40/421; A61P 35/00; C12N 2501/727; C12N 2510/00

(57) ABSTRACT

Provided herein are methods to improve the therapeutic efficacy of double negative T cells (DNTs) and DNTs that have been modified to express one or more chimeric antigen receptor (CAR) molecules, comprising contacting the DNTs with a PI3Kδ inhibitor such as Idelalisib. Also provided are methods to treat cancer using the enhanced DNTs produced by the methods disclosed herein.

This application claims the benefit of U.S. Provisional Application No. 63/357,120 filed Jun. 30, 2022, the contents of which are incorporated herein by reference.

### FIELD

The disclosure relates to methods to produce enhanced DNTs that have increased persistence and methods to treat cancer using the enhanced DNTs.

### BACKGROUND OF THE INVENTION

AML is the most common form of adult acute leukemia with a poor overall 5-year survival rate of 30-45% for patients 60 years old or younger and 10-15% for elderly patients over 60 years of age [1,2]. Although induction chemotherapy is effective, high relapse rate and refractory disease contribute to the poor patient outcome. Currently, allogeneic hematopoietic stem cell transplantation (allo-HSCT) is the only form of treatment with sustained curative potential for chemotherapy-resistant AML patients, where the treatment effect is largely driven by the graft-versus-leukemia (GvL) activity of donor-derived immune cells [1]. However, severe transplant-associated toxicities, such as graft-versus-host disease (GvHD), hinder its clinical benefit for patients [3].

Utilizing the potency of GvL effect, immune cell-based therapy has been an active area of research to provide novel treatment options for high-risk AML patients. Of those, adoptive cellular therapy (ACT) using chimeric antigen receptor (CAR)-T cells and NK cells have demonstrated promising outcomes in early phase clinical trials [4-9]. However, only small cohorts of patients were treated with CAR-T cells, and, while effective, severe treatment-associated toxicities were observed [4-7]. In contrast, NK cell therapy was shown to be safe in multiple trials, but its clinical benefit was variable and largely confined as a consolidation therapy to prevent disease relapse, rather than to treat relapsed or refractory patients [1,8,9]. Also, high treatment-associated costs and difficulties in large scale manufacturing as well as poor persistence are some of the known logistical challenges of CAR-T and NK therapies, respectively [1,7].

Double Negative T (DNT) cells are a rare subset of mature peripheral T lymphocytes that express CD3 in the absence of CD4 and CD8 expression. Murine DNTs have been shown to rescue mice from lethal doses of leukemia, and a correlative study showed that allo-HSCT patients with better DNT recovery have lower risk of disease relapse [10,11]. To that end, methods have been developed to expand DNTs ex vivo and it has been demonstrated that both patient- and donor-derived DNTs not only induce anti-leukemic activity against AML but also target array of solid and hematological cancers by reducing tumour burden in various xenograft models without causing off tumour toxicities [12-15]. Interestingly, ex vivo expanded human DNTs exhibit an innate immune cell-like behaviour, where DNTs recognize cancer cells in an antigen non-specific manner using receptors such as NKG2D, DNAM-1, and TRAIL [13,14,16]. Importantly, it has been demonstrated that donor-derived DNTs fulfill the requirements of an off-the-shelf allogeneic T cell therapy without any genetic modifications. In particular, DNTs lack of off-tumor activity such as GvHD and are resistant to host-versus-graft rejection [13, 17]. Recently, a phase I/IIa clinical trial using allogeneic DNTs to treat AML patients that relapsed after allo-HSCT was completed (ChiCTR-IPR-1900022795). While these patients had extremely poor prognoses, DNT-treatment achieved a 63.6% response rate with a 1-year overall survival of 40.6%. Further, DNTs did not induce any severe treatment-associated toxicity [18]. While these findings make DNT therapy an attractive form of ACT, the persistence and durability of DNT-mediated responses are less well characterized.

The advantage of cell-based therapy as a living drug stems from the persistence of infused cells which mediate the treatment effect for a prolonged duration. Numerous studies consistently demonstrate the importance of infused cell persistence for favorable outcomes [19-25]. It has been demonstrated that ex vivo expanded human DNTs can persist in naïve immune deficient mice for at least 14-21 days [17,26]. However, studies with a longer follow up have not been conducted. Further, the persistence of DNTs in leukemia-bearing hosts may differ from naïve mice as leukemic blasts may provide stimulatory or inhibitory signals that affect the persistence of DNTs [27]. There remains a need for DNTs with improved persistence to enhance the therapeutic efficacy of DNTs.

### SUMMARY

It is demonstrated herein that contacting DNTs with the PIK3δ inhibitor Idelalisib improves the persistence of the DNTs.

Accordingly, one aspect of the present disclosure provides a method of enhancing the therapeutic efficacy of DNTs, comprising contacting DNTs with a PIK3δ inhibitor to produce enhanced DNTs.

In an embodiment, the DNTs comprise or consist of autologous cells. In an embodiment, the DNTs comprise or consist of allogenic cells.

In an embodiment, the PIK3δ inhibitor is selected from the group consisting of idelalisib (Ide), PI-3065 and AZD8835. In an embodiment, the PIK3δ inhibitor is Idelalisib.

The method of contacting DNTs with a PIK3δ inhibitor as described herein produces enhanced DNTs that have a number of characteristics that make the cells more effective for the treatment of cancer. For example, in an embodiment, the enhanced DNTs exhibit anti-tumor activity. In an embodiment, the enhanced DNTs exhibit increased persistence.

In an embodiment, contacting the DNTs with the PIK3δ inhibitor promotes an early memory status of the DNTs. In an embodiment, contacting the DNTs with the PIK3δ inhibitor prevents exhaustion of the DNTs. In an embodiment, contacting the DNTs with the PIK3δ inhibitor improves viability of DNTs.

In an embodiment, the method comprises contacting the DNTs with a concentration of Ide of between about 0.1 μM and about 5.0 μM. In another embodiment, the concentration of Ide is between about 0.1 μM and about 3.0 μM. In yet another embodiment, the concentration of Ide is between about 0.5 μM and about 2.0 μM. In a further embodiment, the concentration of Ide is about 1.0 μM.

In an embodiment, the method comprises contacting the DNTs with Ide for at least about 3 days, at least about 5 days, at least about 10 days, at least about 15 days or at least about 20 days.

In an embodiment, the DNTs are contacted with Ide starting at day 0 of culture or within 7 days of expansion, wherein total expansion is from 10-20 days.

In an embodiment, the method further comprises removing some or all of the PIK3δ inhibitor from contact with the enhanced DNTs.

In an embodiment, the DNTs are CAR-DNTs. In an embodiment, the DNTs are CD4-CAR-DNTs.

In another aspect, the present disclosure provides a population of enhanced DNTs produced by a method described herein. In an embodiment, the enhanced DNTs exhibit an increased persistence as compared to control DNTs that have not been contacted with the PIK3δ inhibitor. In an embodiment, the enhanced DNTs exhibit an increased anti-tumor activity as compared to control DNTs that have not been contacted with the PIK3δ inhibitor. In another embodiment, the enhanced DNTs exhibit an early memory status as compared to control DNTs that have not been contacted with the PIK3δ inhibitor. In yet another embodiment, the enhanced DNTs exhibit a decrease in T cell exhaustion as compared to control DNTs that have not been contacted with the PIK3δ inhibitor. In a further embodiment, the enhanced DNTs exhibit an increase in DNT viability as compared to control DNTs that have not been contacted with the PIK3δ inhibitor.

In another aspect, there is provided a composition comprising the enhanced DNTs produced according to a method disclosed herein and a pharmaceutically acceptable carrier.

In yet another aspect, the present disclosure provides the use of the enhanced DNTs or the compositions described herein for the treatment of cancer in a subject in need thereof. Also provided is a method of treating cancer in a subject in need thereof, the method comprising administering to the subject enhanced DNTs or a composition as described herein. Further provided is the use of the enhanced DNTs described herein in the manufacture of a medicament for treating cancer in a subject in need thereof. Even further 10 provided is the enhanced DNTs or the compositions described herein for use in the treatment of cancer in a subject in need thereof.

In an embodiment, the cancer is leukemia, optionally acute myeloid leukemia (AML).

In an embodiment, the enhanced DNTs are produced from CD4-CAR-DNTs, and the cancer is T cell leukemia. In an embodiment, the enhanced DNTs are CD4-CAR-DNTs and the cancer is T cell lymphoma. In an embodiment, the enhanced DNTs are CD19-CAR-DNTs and the cancer is B cell leukemia. In an embodiment, the enhanced DNTs are CD19-CAR-DNTs and the cancer is B cell lymphoma.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIGS. 1A-1B. BET bromodomain inhibitor, JQ-1, does not affect the memory status of DNTs. (FIG. 1A) Representative flow plots showing memory status. Memory status of ctrl-DNT and Ide-DNT on day 14 of expansion determined by CD45RA and CD62L expression. T_{naive/SCM}=CD45RA⁺CD27⁺; T_{CM}=CD45RA⁻CD27⁺; T_{EM}=CD45RA⁻CD27⁻; T_{effector}=CD45RA⁺CD27⁻. (FIG. 1B) MFI values of T cell memory markers, CD27, CD45RA, and CD62L, on DNTs expanded in the presence of increasing concentrations of JQ-1 on day 14 of expansion.

FIGS. 2A-2B. BET bromodomain inhibitor, JQ-1, does not affect the memory status of DNTs. (FIG. 2A) Representative flow plots showing memory status. Memory status of ctrl-DNT and Ide-DNT on day 17 of expansion determined by CD45RA and CD62L expression. T_{naive/SCM}=CD45RA⁺CD27⁺; T_{CM}=CD45RA⁻CD27⁺; T_{EM}=CD45RA⁻CD27⁻; T_{effector}=CD45RA⁺CD27⁻. (FIG. 2B) MFI values of T cell memory markers, CD27, CD45RA, and CD62L, on DNTs expanded in the presence of increasing concentrations of JQ-1 on day 17 of expansion.

FIGS. 3A-3B. BET bromodomain inhibitor, JQ-1, does not affect the memory status of DNTs. (FIG. 3A) Representative flow plots showing memory status. Memory status of ctrl-DNT and Ide-DNT on day 20 of expansion determined by CD45RA and CD62L expression. T_{naive/SCM}=CD45RA⁺CD27⁺; T_{CM}=CD45RA⁻CD27⁺; T_{EM}=CD45RA⁻CD27⁻; T_{effector}=CD45RA⁺CD27⁻. (FIG. 3B) MFI values of T cell memory markers, CD27, CD45RA, and CD62L, on DNTs expanded in the presence of increasing concentrations of JQ-1 on day 20 of expansion.

FIGS. 4A-4E. DNT persistence and anti-leukemic activity in leukemia bearing hosts. (FIG. 4A) Schematic diagram of patient-derived xenograft (PDX) model used. Sublethally irradiated NSG mice were infused with 2-3×10⁶ primary AML patient sample on day 0. On day 17 and 20, mice were infused with 2×10⁷ DNTs. Mice were sacrificed and DNT and AML engraftment were assessed in various tissues on day 37 or day 70. (FIGS. 4B-4C) NSG mice engrafted with a primary AML sample (090543) were treated with PBS or DNT as described in FIG. 4A. Number of viable DNTs engrafted in bone marrow, spleen, blood, liver, and lung were assessed on day 37, which was 17 days post DNT infusion (FIG. 4B). Number of DNTs detected in mice peripheral blood from day 14 to 45 days post DNT infusion (FIG. 4C). Each symbol represents individual mice, and error bars represent SEM. (FIGS. 4D-4E) NSG mice engrafted with a primary AML sample (130624) were treated with PBS or DNT as described in above. Mice were sacrificed and DNT and AML engraftment were assessed in various tissues on day 70, which was 50 days post-treatment. Representative flow plots showing AML engraftment (CD45^{low+}CD3⁻) and DNT engraftment (CD45^{high+}CD3⁺) from untreated and DNT treated groups (FIG. 4D). The representative frequency and number of viable AML cells in untreated (n=5) and DNT-treated (n=7) mice bone marrow and spleen (FIG. 4E). Each symbol represents individual mice, the horizontal bars represent the mean and the error bars represent SEM. The result is representative of 2 similar experiments with different AML primary blasts.

FIGS. 5A-5D. Ide significantly enhances the anti-leukemic function of DNTs in PDX model. (FIG. 5A) Schematic diagram of patient-derived xenograft (PDX) model used. Sublethally irradiated NSG mice were infused with 2-3×10⁶ primary AML patient sample on day 0. On day 17 and 20, mice were infused with 2×10⁷ ctrl-DNTs or Ide-DNTs. Mice were sacrificed and AML engraftment were assessed in various tissues on day 24 or day 34. (FIG. 5B) NSG mice engrafted with a primary AML sample (090543) were treated with PBS, ctrl-DNT or Ide-DNT as described in FIG. 4A. AML engraftment and counts in bone marrow were assessed on day 34. Representative flow plots show bone marrow AML engraftment (CD45⁺CD33⁺). Dot plot shows the frequency and number of AML cell engrafted in the bone marrow. Horizontal line represents the mean of BM AML engraftment level, each symbol represents individual mice, and error bars represent SEM. The results shown are representative of two independent experiments. (FIG. 5C) Percent reduction in AML engraftment from two patient-derived xenograft experiments performed using different primary AML patient samples (090543 and 130624). Horizontal bar represents the mean of reduction in AML engraftment level in BM on day 34 relative to the untreated group in ctrl-DNT and Ide-DNT treated groups. Each symbol represents individual mice and error bars represent SEM. (FIG. 5D) Sublethally irradiated (250 cGy) NSG mice were engrafted with one of 4 primary AML samples (sample ID: 150935, 130578, 090543, and 130624; 2-3×10⁶ cells/mouse). Subsequently, mice were treated with PBS or 1.5-2×10⁷ cells per infusion of ctrl-DNTs or Ide-DNTs on day 17 and 20. For PDX model using sample ID 150935 and 130578, BM AML engraftment was assessed on day 24, which was 4 days post-treatment. For sample ID 090543 and 130624, BM AML engraftment was assessed on day 34, which was 14 days post-treatment. Each group had 4-8 mice. Horizontal bar represents the mean of BM AML engraftment level normalized to untreated group, and error bars represent SEM.

FIGS. 6A-6F. Ide promotes an early memory subset of DNTs that have better proliferative capacity. (FIG. 6A) Representative flow plots showing memory status. Memory status of ctrl-DNT and Ide-DNT on day 17 of expansion determined by CD45RA and CD62L expression. T_{naive/SCM}=CD45RA⁺CD62L⁺; T_{CM}=CD45RA⁻CD62L⁺; T_{EM}=CD45RA⁻CD62L; T_{effector}=CD45RA⁺CD62L⁻. (FIG. 6B) Frequency of DNTs in early memory subset T_{CM/SCM} (CD45RA⁺/CD62L⁺ and CD45RA⁻CD62L⁺; left), T_{EM} (CD45RA⁻CD62L⁻; middle) and T_{EFF} (CD45RA⁺CD62L⁻; right) from day 7 to day 17 of ex vivo DNT expansion culture. (FIG. 6C) T_{CM/SCM} cell number of ctrl-DNT and Ide-DNT on day 17 of ex vivo DNT expansion culture. Each dot represents DNTs from one donor. The cell number is normalized to day 0 DNT number. (FIG. 6D) Relative expression of memory associated genes, TCF7, LEF1, SELL and IL7R, in ctrl-DNT and Ide-DNT determined by qPCR. The expression shown is relative to the expression of housekeeping gene, HPRT. Each paired symbols represent DNTs from one donor. (FIG. 6E) Expansion of ctrl-DNT and Ide-DNTs in the presence of DNT-susceptible AML cell line, OCI-AML3. Expansion fold relative to the number of DNTs at the start of co-culture was determined after 2 days and 8 days (Left). Absolute number of viable DNTs from each treatment group after co-culture with OCI-AML3 (Right). The results shown are representative of three independent experiments. (FIG. 6F) Memory status of DNTs 8 days post co-culture with OCI-AML3 determined by flow-cytometry. The result shown is representative of three independent experiments.

FIGS. 7A-7G. Ide improves fitness of DNTs by inhibiting exhaustion and enhancing viability. (FIG. 7A) Expression of exhaustion markers, LAG3 and TIM3, on unstained, ctrl-DNT and Ide-DNT. Representative flow plot is shown (Left). The numbers represent the MFI value (Right). (FIG. 7B) The summary of results comparing relative MFI of LAG3 and TIM3 on ctrl-DNT and Ide-DNT from 5 donors. Horizontal bars represent the mean relative MFI and error bars show SEM. (FIG. 7C) Expression of exhaustion associated transcription factor gene, TOX, relative to the expression of housekeeping gene, HPRT, determined by qPCR. Each paired symbol represents DNTs from one individual. (FIG. 7D) Production of TNFα and IFNγ by ctrl-DNT and Ide-DNT with or without ex vivo stimulation. Ctrl-DNT and Ide-DNT were treated with protein transport inhibitor, followed by stimulation using anti-CD3/CD28 activation beads for 4 hours. Subsequently, cells were intracellularly stained with anti-TNFα and anti-IFNγ antibodies, and the expression levels were determined by flow cytometry. Representative flow plot is shown (Left). The numbers represent the frequency in each gate. Bar graph shows the percentage of ctrl-DNT and Ide-DNT expressing TNFα, IFNγ, and TNFα and IFNγ (Right). The result shown is a summary of three experiments. (FIG. 7E) Frequency of viable ctrl-DNTs and Ide-DNTs on day 17 of expansion. The cell viability was determined with Annexin V and 7-AAD staining using flow cytometry. Flow plot shows a representative image (Left). Dot plot shows summary of results using DNTs from five different donors (Right). Horizontal bars represent the mean, and the error bar shows SEM. (FIG. 7F) Viability of ctrl-DNTs and Ide-DNTs after co-culture with primary AML cells from 7 different patients. Ctrl-DNTs and Ide-DNs were co-incubated with primary AML cells at 4:1 effector-to-target ratio for 2 hours. Subsequently, the viability of DNTs (gated on CD45^{high} CD3⁺CD33/CD34⁻) were determined using flow cytometry. (FIG. 7G) Expression of anti-apoptotic gene, BCL2, on ctrl-DNT and Ide-DNTs relative to the expression of housekeeping gene, HPRT, was determined by qPCR. Each paired symbol represents DNTs from one donor.

FIGS. 8A-8L. Ide prolongs persistence of DNT and promotes a durable anti-leukemic effect in vivo. Schematic diagram of patient-derived xenograft (PDX) model used. (FIG. 8A) Sublethally irradiated NSG mice were infused with 2-3×10⁶ primary AML patient sample on day 0. On day 17 and 20, mice were infused with 2×10⁷ ctrl-DNTs or Ide-DNTs. Mice were sacrificed and DNT persistence and AML engraftment were assessed in various tissues on day 34 or day 100. (FIGS. 8B-8E) NSG mice engrafted with primary AML sample (090543) were treated with PBS (n=8), ctrl-DNT (n=8), or Ide-DNT (n=9), on day 17 and 20 post AML infusion. Subsequently, DNT engraftment in BM (CD45^{high}CD3⁺; B), the frequency (FIG. 8C) and the number of (FIG. 8D) of T_{CM} (CD45^{high}CD3⁺CD45RA⁻CD62L⁺), T_{EM} (CD45^{high}CD3⁺CD45RA⁻CD62L⁻), and T_{EFF} (CD45^{high}CD3⁺CD45RA⁺CD62L⁻) and expression level of exhaustion markers (FIG. 8E), LAG3, TIM3, and CTLA4, within the DNT population were determined on day 34, which was 14 days post-treatment using flow cytometry. Each dot represents one mouse. The result is representative of 2 similar experiments with different AML primary blasts. (FIGS. 8F-8H) Sublethally irradiated NSG mice were engrafted with primary AML sample (090517), followed by PBS, ctrl-DNT, or Ide-DNT treatment on day 17 and 20. Subsequently, the frequency (left) and number (right) of DNTs in peripheral blood were determined between day 21-74 post last DNT infusion in ctrl-DNT and Ide-DNT using flow cytometry (FIG. 8F). Representative flow plots show DNT persistence from peripheral blood on day 94, which was 74 days post-treatment (FIG. 8G). Number of viable DNT engrafted in bone marrow, spleen, blood, liver, and lung on day 100, which was 80 days post-treatment. Each dot represents one mouse (FIG. 8H). (FIG. 8I) Correlative analysis of DNT and AML cell numbers in bone marrow (left) and spleen (right) in AML-engrafted mice treated with ex vivo expanded DNTs. The results are representative of three independent xenograft experiments done using AML cell line, MV4-11 (n=1) and two primary AML patient samples. (FIG. 8J) NSG mice engrafted with primary AML sample (090543) were treated with PBS, ctrl-DNT, and Ide-DNT on day 17 and 20. Subsequently, the AML level in mice peripheral blood relative to the untreated group was determined on day 45, 52, 58, and 65. (FIGS. 8K-8L) NSG mice engrafted with primary AML sample (090517) were treated with PBS (n=5), ctrl-DNT (n=7), and Ide-DNT (n=6) on day 17 and 20. Subsequently, % AML engraftment relative to untreated were determined on day 100, which was 80 days post-treatment (FIG. 8K) Representative flow plots show AML engraftment (CD45^{low+}CD33⁺) from BM, spleen, liver and lung (FIG. 8L).

FIG. 9. DNT persistence in leukemic bearing mice. NSG mice engrafted with a primary AML sample (090543) were treated with PBS (top panels) or DNT (bottom panels) as described in FIG. 4A. Cells are gated within DAPI⁻CD45⁺. Representative flow plots showing AML (DAPI⁻CD45⁺CD3⁻CD33⁺) and DNT (DAPI⁻CD45⁺CD3⁺CD33⁻) engraftment from untreated or DNT treated group on day 37, which was 17 days post DNT infusion.

FIGS. 10A-10B. Expansion of DNTs in the varying concentrating of Ide. (FIGS. 10A-10B) DNTs expanded in varying concentration of Ide (0.3-10 μM) or vehicle control from day 0 to day 21. Cell number of DNTs from day 7 to day 21 of ex vivo DNT expansion culture (FIG. 10A). % viable (FSC/SSC_{live lymphocyte}×AnnexinV⁻) DNTs on day 21 (FIG. 10B).

FIG. 11. Ide treated DNTs significantly reduce AML engraftment level in spleen. NSG mice engrafted with a primary AML sample (090543) were treated with PBS, ctrl-DNT or Ide-DNT as described in FIG. 4A. AML engraftment and counts in spleen were assessed on day 34. Representative flow plots show spleen AML engraftment (CD45⁺CD33⁺; Top). Dot plot shows the frequency and number of AML cell engrafted in the spleen (Bottom). Horizontal line represents the mean of AML engraftment level and each symbol represents individual mice, and error bars represent SEM. The results shown are representative of two independent experiments.

FIG. 12. ctrl-DNTs and Ide-DNTs do not cause tissue damage. NSG mice engrafted with primary AML sample (090543) were treated with PBS (n=8), ctrl-DNT (n=8), or Ide-DNT (n=9) on day 17 and 20 post AML infusion. Subsequently, tissue from liver and lung were fixed on day 34 for H&E staining. Representative H&E-stained slides of liver (Top) with ×400 magnification and lung (Bottom) with ×100 magnification from untreated (Left), ctrl-DNT (Middle) and Ide-DNT (Right) group. PV-portal vein; ALV-alveoli; BR-bronchioles.

FIG. 13. Ide treatment reduce anti-leukemic function of DNTs in vitro. Cytotoxicity Assay with ctrl-DNT or Ide-DNT as effector cells and AML cell lines, OCI-AML3 and MV4-11, or primary blasts, 141005 and 140372, as target cells. 14-17 day ex vivo expanded ctrl-DNT and Ide-DNT were co-cultured with target cells for 2 hours. Subsequently, cells were stained with Annexin V, CD3, CD33/CD34. Viability of targets (gated on CD3⁻CD33/CD34⁺) was determined using flow cytometry to calculate % specific killing by DNTs.

FIGS. 14A-14C. Ide promotes early memory subset of DNTs. Representative flow plots showing T_{SCM} (CD45RA⁺CD62L⁺) cells expressing CD95 using day 14 ex vivo expanded DNTs (FIG. 14A). Frequency of DNTs in early memory subset T_{CM/SCM} (CD45RA⁺CD62L⁺ and CD45RA⁻CD62L⁺) on day 17 of expanded DNTs in 0, 0.3 and 1 μM of Ide (FIG. 14B). Expression of transcription factor gene important for effector T cell differentiation, BATF, relative to the expression of housekeeping gene, HPRT, determined by qPCR. Each paired symbols represents DNTs from one individual (FIG. 14C).

FIG. 15. Ide enhances proliferative capacity of DNT after encountering AML in vitro. Expansion of ctrl-DNT and Ide-DNTs in the presence of DNT-susceptible AML cell line, OCI-AML3. Flow plot shows CFSE changes in DNT population (CD3⁺CD33⁻) on day 0 and day 7 after co-culture (left). Fold reduction in MFI value of CFSE was determined on 0, 3 days and 7 days post co-culture from each treatment group (right).

FIGS. 16A-16B. Idelalisib prolongs persistence of DNTs in vivo. (FIG. 2. 16A-16B) NSG mice engrafted with primary AML sample (090543) were treated with PBS (n=8), ctrl-DNT (n=8), or Ide-DNT (n=9) on day 17 and 20 post AML infusion. Subsequently, Spleen (FIG. 16A) and liver (FIG. 16B) engraftment of DNT (CD45^{high}CD3⁺) were determined on day 34, 14 days post DNT infusion using flow cytometry. Representative flow plot shows DNT engraftment (CD45^{high}CD3⁺).

FIG. 17. Ide prolongs the persistence of DNT. NSG mice engrafted with primary AML sample (090517) were treated with PBS (n=5), ctrl-DNT (n=7), or Ide-DNT (n=6), on day 17 and 20 post AML infusion. Representative flow plots show DNT engraftment (CD45^{high}CD3⁺) from BM, spleen, liver and lung that were determined on day 100, which was 80 days post DNT infusion using flow cytometry.

FIG. 18. Idelalisib inhibits activation of DNTs. Frequency of CD69 expression from day 7, 10 and 17 ex vivo expanded DNTs in 0, 1 and 5 μM of de.

FIGS. 19A-19C. Lowering soluble OKT3 concentration did not affect differentiation but cytotoxicity. (FIGS. 19A-19C) DNTs expanded in 0.1, 0.05 and 0.025 ug/mL of soluble OKT3 from day 7 to day 17. Representative flow plots showing memory status of DNTs on day 17 of expansion (FIG. 19A). T_{SCM}=CD45RA⁺CD62L⁺; T_{CM}=CD45RA⁻CD62L⁺; T_{EM}=CD45RA⁻CD62L; T_{effector}=CD45RA⁺CD62L. Expression of exhaustion markers, LAG3 and TIM3 MFI value of DNTs on day 17 of expansion (FIG. 19B). Cytotoxicity Assay with DNTs as effector cells and AML cell lines, OCI-AML3 and MV4-11 as target cells (FIG. 19C). Effector cells were co-cultured with target cells with 0.25 to 1, effector to target ratio, overnight. Subsequently, cells were stained with 7AAD, AnnexinV, CD3, CD33. Viability of targets (gated on CD3⁻CD33⁺) were determined using flow cytometry to calculate % specific killing by DNTs.

FIG. 20. Idelalisib did not change the expression of REGNASE-1. Expression of REGNASE-1 relative to the expression of housekeeping gene, HPRT, was determined by qPCR. Each paired symbols represents DNTs from one individual.

FIG. 21. CAR4-Ide-DNTs show better memory phenotype compared to CAR4-DNTs. Frequency of naïve (CD45RA+CD62L+), central memory (CD45RA−CD62L+), effector memory (CD45RA−CD62L−), and terminally differentiated effector memory (CD45RA+CD62L−) cells on different days of CAR4-DNTs cultured in the presence or absence of Ide.

FIGS. 22A-22B. Ide-treatment improves the ability of CAR4-DNTs to target T-ALL in re-challenge model. (FIG. 22A) CAR4-DNTs or CAR4-Ide-DNTs were cocultured with T-ALL cell line, CCRF-CEM, for 2 hours at a 1:1 effector-to-target ratio. Percent specific killing of the target is shown. Bars represent mean of technical triplicates, and error bars represent SEM. (FIG. 22B) CAR4-DNTs (circle) and CAR4-Ide-DNTs (square) were cocultured with CCRF-CEM at a 4:1 effector-to-target ratio. Every 24 hours following coculture, percent specific killing of the target was measured, and fresh target cells were added to the coculture at original effector-to-target ratio. Dots represent mean percent specific killing of triplicates and error bars represent SEM.

FIGS. 23A-23E. CAR4-Ide-DNTs significantly improved the survival of T-ALL engrafted mice compared to CAR4-DNTs. (FIG. 23A) Schematic outline of in vivo experiments. NSG mice were sublethally irradiated (250 cGy) on day −1 and intravenously (i.v.) injected with 2×10⁵ to 5×10⁵ CCRF-CEM cells on day 0. On days 3, 7, and 10, mice were intravenously treated with PBS, 2×10⁶ CAR⁺ non-treated CAR4-DNTs or 2×10⁶ CAR⁺ Ide-treated CAR4-DNTs. All mice received intraperitoneal (i.p.) injections of IL-2 once per week. Mice were sacrificed when they reached the end point of the survival experiment, and cells were harvested from the bone marrow, spleen, liver, and lungs. CCRF-CEM cells were identified via staining with anti-CD45 and anti-HLA-A2 antibodies. (FIG. 23B) Kaplan-Meier curve showing the percent survival of CCRF-CEM-engrafted mice treated with PBS (n=14), 2×10⁶ CAR⁺ non-treated CAR4-DNTs (n=12), 2×10⁶ CAR⁺CAR4-DNTs (n=5). Log-rank test was used for statistical analysis. (FIG. 23C) Representative flow plots for HLA-A2 and CD45 staining of blood cells from PBS-, CAR4-DNT-, and Ide-CAR4-DNT-treated mice on day 26 post CCRF-CEM engraftment. An HLA-A2-mistmatched donor was used to manufacture CAR4-DNTs in order to distinguish from CCRF-CEM cells. (FIG. 23D) CCRF-CEM cell counts in the peripheral blood of mice after treatment with PBS, CAR4-DNTs and Ide-CAR4-DNTs (n=2 for each treatment group). Each dot represents CCRF-CEM cell count per mL of blood in one mouse, horizontal lines represent the mean, and the error bars represent SEM. (FIG. 23E) Representative flow plots for HLA-A2 and CD4 staining of lung cells from untreated, CAR4-DNT-treated, and Ide-CAR4-DNT-treated mice at the end point of the survival experiment. Flow plots are gated on human CD45⁺ cells and numbers represent percentage of CD45⁺ cells. An HLA-A2-mistmatched donor was used to manufacture CAR4-DNTs in order to distinguish from CCRF-CEM cells.

FIGS. 24A-24C Effect of different Pi3K-delta inhibition schedule on memory formation on DNTs. DNTs were expanded in the absence or presence of Ide (1 μM) from day 0-14, 3-14, 0-7, and 7-14 of culture. The frequency of naïve, central memory, effector memory, and terminally differentiated effector memory DNTs were determined on day 14 of culture. (FIG. 24A) show representative flow plot of DNTs expanded in each group. (FIG. 24B) show the frequency of CD62L positive DNTs from two different donors in each treatment group on different days. (FIG. 24C) Representative flow plot of DNTs in different memory status when expanded in the absence or presence of Ide from day 0-10 and day 1-10.

### DETAILED DESCRIPTION

Double negative T cell (DNT) is a unique subset of T cells with potent anti-cancer potential. Previously, DNT therapy has been shown to effectively target leukemia and lymphoma cells in patient-derived xenograft (PDX) models. Further, a recently completed phase I clinical study (Chinese Trial Registry Clinical www.chictr.org.cn: ChiCTR-IPR-1900022795) demonstrated the safety, feasibility, and potential efficacy in AML patients that relapsed after allogeneic hematopoietic stem cell transplantation (Tang B et al. Allogeneic double-negative T cell therapy for relapsed acute myeloid leukemia patients post allogeneic hematopoietic stem cell transplantation: A first-in-human phase I study. Am J Hematol. 2022 July; 97(7): E264-E267.). However, the persistence and durability of DNT-mediated anti-leukemic response is less well understood.

Early memory phenotype of infused cells is an important dictator for immune cell persistence. JQ1, which is an inhibitor of bromodomain and extra-terminal motif (BET) proteins, was shown to induce memory phenotype in conventional T cells (Kagoya et al. J Clin Invest. 2016 Sep. 1; 126 (9): 3479-3494). However, as demonstrated herein, JQ1 does not induce a similar memory phenotype in DNTs despite its effect on memory phenotype in conventional T cells.

In contrast to JQ1, the present inventors have demonstrated that Idelalisib pretreatment of DNTs results in improved persistence of DNTs.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the term “DNTs”, “DNT cells” or “double negative T cells” refers to a rare subset of mature peripheral T lymphocytes that expresses CD3 in the absence CD4 and CD8 expression.

As used herein, the term “ex vivo expansion” refers to in vitro culturing of DNTs that have been collected ex vivo from a subject.

The term “contacted” or “contacting” as used herein refers to any method of exposing DNTs to a PI3Kδ inhibitor during culture/expansion to produce enhanced DNTs. “Contacting” includes “incubating”, “exposing” and “pretreating” and does not imply any specific time or temperature requirements, unless otherwise indicated.

As used herein, the term “PI3Kδ inhibitor” refers to an agent, compound, or composition that reduces or abolishes the activity of PI3Kδ, which is one of the isoforms of phosphoinositide 3-kinases (PI3Ks).

As used herein, the term “Idelalisib” or “Ide” refers to the compound 5-fluoro-3-phenyl-2-[(1S)-1-(7H-purin-6-ylamino) propyl]quinazolin-4-one having the structure:

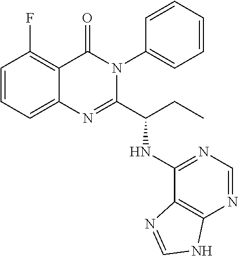 [SMILES: CC[C@H](Nc1ncnc2[nH]cnc12)c1nc2cccc(F)c2c(=O)n1-c1ccccc1]

As used herein, the term “PI-3065” refers to the compound 6-[[4-(cyclopropylmethyl)-1-piperazinyl]methyl]-2-(5-fluoro-1H-indol-4-yl)-4-(4-morpholinyl)-thieno[3,2-d]pyrimidine having the structure:

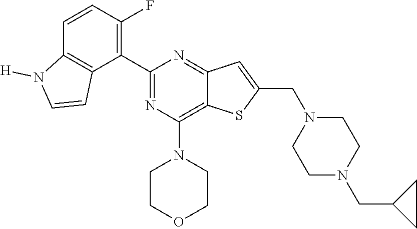 [SMILES: Fc1ccc2[nH]ccc2c1-c1nc(N2CCOCC2)c2sc(CN3CCN(CC4CC4)CC3)cc2n1]

As used herein, the term “AZD8835” refers to the compound 1-[4-[5-[5-amino-6-[5-(1,1-dimethylethyl)-1,3,4-oxadiazol-2-yl]-2-pyrazinyl]-1-ethyl-1H-1,2,4-triazol-3-yl]-1-piperidinyl]-3-hydroxy-1-propanone having the structure:

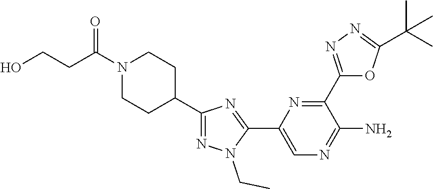 [SMILES: CCn1nc(C2CCN(C(=O)CCO)CC2)nc1-c1cnc(N)c(-c2nnc(C(C)(C)C)o2)n1]

As used herein, the term “cancer” refers to one of a group of diseases caused by the uncontrolled, abnormal growth of cells that can spread to adjoining tissues or other parts of the body. Cancer or tumor cells can form a solid tumor, in which the cancer cells are massed together, or exist as dispersed cells, as in a hematological cancer such as leukemia.

The term “cancer cell” or “tumor cell” refers to a cell characterized by uncontrolled, abnormal growth and the ability to invade another tissue or a cell derived from such a cell. Cancer cells include, for example, a primary cancer cell obtained from a patient with cancer or a cell line derived from such a cell. In an embodiment, the cancer cell is a hematological cancer cell, such as a leukemic cell or a lymphoma cell.

The term “tumor” as used herein refers to a collection of cancer cells.

The term “anti-tumor activity” as used herein refers to any activity of killing tumor cells and/or inhibiting tumor growth.

The term “leukemia” as used herein refers to any disease involving the progressive proliferation of abnormal leukocytes found in hemopoietic tissues, other organs and usually in the blood in increased numbers. “Leukemic cells” refers to leukocytes characterized by an increased abnormal proliferation of cells. Leukemic cells may be obtained from a subject diagnosed with leukemia.

The term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells. Pre-leukemic conditions such as myelodysplastic or myeloproliferative syndromes may also develop into AML.

The term “subject” as used herein includes all members of the animal kingdom including mammals, and suitably refers to humans. Optionally, the term “subject” includes mammals that have been diagnosed with cancer or are in remission. In an embodiment, the term “subject” refers to a human having, or suspected of having, cancer.

The term “treat”, “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment.

As used herein, the phrase “effective amount” or “therapeutically effective amount” means an amount effective, at dosages and for periods of time necessary to achieve the desired result. For example, in the context or treating cancer, an effective amount is an amount that, for example, induces remission, reduces tumor burden, and/or prevents tumor spread or growth of cancer cells compared to the response obtained without administration of the compound. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

As used herein, the term “CAR-DNT cells” or “CAR-DNTs” refers to double negative T-cells (“DNT cells” or “DNTs”) that have been modified to express one or more chimeric antigen receptor (CAR) molecules. Such CAR-DNTs may be described as being derived from DNTs.

As used herein, the term “CAR4-DNTs” or “CD4-CAR-DNTs” refers to CAR-DNTs that target the CD4 antigen.

As used herein, the term “CAR” refers to a chimeric antigen receptor. A CAR molecule can comprise an extracellular antigen binding domain, a transmembrane domain, and an intracellular domain.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

As used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the description, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the description and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “about” as used herein means plus or minus 0.1 to 20%, 5-20%, 10-20%, 10%-15%, 5-10%, or 5% of the number to which reference is being made.

It should also be understood that, in certain methods described herein that include more than one step or act, the order of the steps or acts of the method is not necessarily limited to the order in which the steps or acts of the method are recited unless the context indicates otherwise.

Although any methods and materials similar or equivalent to those described herein can also be used in the practice or testing of the present disclosure, examples of methods and materials are now described.

### II. Methods of Producing Enhanced DNTs

In an embodiment, there is provided a method of enhancing the therapeutic efficacy of DNTs comprising contacting the DNTs with a PI3Kδ inhibitor during ex vivo expansion of the DNTs to produce enhanced DNTs.

DNTs can be obtained by enriching using CD4 and CD8-depletion antibody cocktails. In an embodiment, the DNTs may be obtained from a sample comprising peripheral blood mononuclear cells (PBMC). In an embodiment, the sample is a blood sample. In another embodiment, the sample is an apheresis sample, or an enriched leukapheresis product such as a leukopak. In yet another embodiment, the sample is a bone marrow sample.

Exemplary methods for isolating and expanding DNTs are described in U.S. Pat. No. 6,953,576 “Method of Modulating Tumor Immunity”, PCT Publication No. WO2007/056854 “Method of Expanding Double Negative T Cells”, and PCT Publication No. WO2016/023134 “Immunotherapy for the Treatment of Cancer” all of which are hereby incorporated by reference in their entirety. DNTs can also be isolated and expanded as described in Example 2.

Any suitable method can be used to expand the DNTs. For example, soluble OKT3 and IL2 may be used. Methods to produce clinical grade DNTs that can be used with the methods disclosed herein is known in the art, for example, Lee J et al. Developing Allogeneic Double-Negative T Cells as a Novel Off-the-Shelf Adoptive Cellular Therapy for Cancer. Clin Cancer Res 1 Apr. 2019; 25 (7): 2241-2253 [17], the content of which is incorporated herein in its entirety.

As demonstrated herein, enhanced DNTs can be produced by contacting DNTs with a PI3Kδ inhibitor during ex vivo expansion.

In an embodiment, the DNTs comprise or consist of autologous cells. In an embodiment, the DNTs comprise or consist of allogenic cells.

In some embodiments, contacting the DNTs with the PI3Kδ inhibitor increases persistence of the DNTs. In some embodiments, contacting the DNTs with the PI3Kδ inhibitor increases anti-tumor activity of the DNTs. In some embodiments, contacting the DNTs with the PI3Kδ inhibitor promotes an early memory status of the DNTs. In some embodiments, contacting the DNTs with the PI3Kδ inhibitor prevents exhaustion of the DNTs. In some embodiments, contacting the DNTs with the PI3Kδ inhibitor improves viability of the DNTs.

An early memory status can for example be indicated by one or more of increased proportion of DNTs with central memory phenotype (T_{CM}), increased proportion of DNTs with stem cell memory phenotype (T_{SCM}), decreased proportion of DNTs with effector memory phenotype (T_{EM}), and/or decreased proportion of DNTs with terminally differentiated effector subtype (T_{EFF}).

Different markers can be used to assess such phenotype. T_{CM} can for example be CD45RA⁻CD62L⁺or CD45RA⁻CD27⁺; T_{SCM} can for example be CD45RA⁺CD62L⁺CD95⁺ or CD45RA⁺CD27⁺; T_{EM} can for example be CD45RA⁻CD62L⁻ or CD45RA⁻CD27⁻; T_{EFF} can for example be CD45RA⁺CD27⁻.

In an embodiment, enhanced DNTs may be distinguished from other DNTs and/or control DNTs on the basis of physiological activity and/or gene expression.

For example, in an embodiment, contacting the DNTs with the PI3Kδ inhibitor increases the proportion of expanded DNTs with central memory phenotype (T_{CM}; CD45RA⁻CD62L⁺) and/or stem cell memory phenotype (T_{SCM}; CD45RA⁺CD62L⁺CD95⁺) as compared to control DNTs that have not been contacted with the PI3Kδ inhibitor.

In an embodiment, contacting the DNTs with the PI3Kδ inhibitor decreases the proportion of expanded DNTs with effector memory phenotype (T_{EM}; CD45RA CD62L⁻) as compared to control DNTs that have not been contacted with the PI3Kδ inhibitor.

In another embodiment, contacting the DNTs with the PI3Kδ inhibitor increases the expression of one or more stem cell memory-associated transcription factor genes as compared to control DNTs that have not been contacted with the PI3Kδ inhibitor. Examples of stem cell memory-associated transcription factor genes include but are not limited to TCF7 and LEF1.

In yet another embodiment, contacting the DNTs with the PI3Kδ inhibitor increases the expression of one or more memory associated genes as compared to control DNTs that have not been contacted with the PI3Kδ inhibitor. Examples of memory associated genes include but are not limited to SELL and IL7R.

In a further embodiment, contacting the DNTs with the PI3Kδ inhibitor decreases the expression of one or more inhibitory receptors on the DNTs as compared to control DNTs that have not been contacted with the PI3Kδ inhibitor. Examples of inhibitory receptors include but are not limited to Lag3 and Tim3.

In yet a further embodiment, contacting the DNTs with the PI3Kδ inhibitor decreases the expression of one or more transcription factor genes that promote T cell exhaustion. Examples of transcription factor genes that promote T cell exhaustion include but are not limited to TOX.

In some embodiments, the enhanced DNTs exhibit increased persistence and/or anti-tumor activity. Characteristics associated with improved persistence of DNTs include, for example, early memory status, improved proliferative capacity, and/or reduced exhaustion.

In some embodiments, the method further comprises removing the PI3Kδ inhibitor from the enhanced DNTs. For example, the PI3Kδ inhibitor can be removed by centrifuging the cell culture to pellet the enhanced DNTs and removing the supernatant. The pelleted enhanced DNTs may be washed one or more times with, for example, PBS.

Any PI3Kδ inhibitor may be used with the methods disclosed herein. In some embodiments, the PI3Kδ inhibitor is selected from the group consisting of Idelalisib (Ide), PI-3065 and AZD8835.

In an embodiment, the PI3Kδ inhibitor is Idelalisib (Ide).

In an embodiment, the DNTs are contacted with Ide during ex vivo expansion at a concentration of between about 0.1 μM and about 5 μM. In an embodiment, the DNTs are contacted with Ide during ex vivo expansion at a concentration of between about 0.1 μM and about 3.0 μM. In another embodiment, the DNTs are contacted with Ide during ex vivo expansion at a concentration of between about 0.5 M and about 2.0 μM. In yet another embodiment, the DNTs are contacted with Ide during ex vivo expansion at a concentration of about 1.0 μM.

In an embodiment, the DNTs are contacted with Ide for at least about 3 days. In another embodiment, the DNTs are contacted with Ide for at least about 5 days. In yet another embodiment, the DNTs are contacted with Ide for at least about 10 days. In a further embodiment, the DNTs are contacted with Ide for at least about 15 days. In yet a further embodiment, the DNTs are contacted with Ide for at least about 20 days.

In an embodiment, the DNTs are contacted with Ide starting at day 0 of culture. In an embodiment, the DNTs are contacted with Ide within 7 days of expansion.

In an embodiment, the DNTs are genetically modified DNTs that express a nucleic acid molecule encoding a chimeric antigen receptor (CAR) that binds to a specific target antigen, also referred to as CAR-DNTs. Any suitable method can be used to modify a DNT cell to produce the CAR-DNTs. For example, the modified DNT cell can be generated by transduction with a vector, plasmid or mRNA comprising a sequence encoding for the CAR.

Other methods to produce the CAR-DNTs are also contemplated. For example, CAR-T cells can be subject to depletion of CD4⁺ and CD8⁺ cells.

In an embodiment, the extracellular binding domain of the CAR binds to a suitable target antigen. The target antigen may be any suitable antigen such as a target that is expressed on the surface of a cancer cell. Suitable antigens include, but are not limited to CD4, CD33, CD19, CD20, CD123 LeY, Mesothelin, EGFR, ROR1, EpCam, MUC1, HER1/2, MET/HGF, neoantigens (driver, non-driver), MAGE family and NY-ESO-1. In an embodiment, the target antigen is CD4. In an embodiment, the target antigen is CD19.

In an embodiment, the DNTs are CD4-CAR-DNTs. In an embodiment, the DNTs are CD19-CAR-DNTs.

### III. Products, Compositions and Kits

In another aspect, there is provided a population of enhanced DNTs produced according to the methods described herein. Also provided are compositions comprising enhanced DNTs as described herein. For example, in an embodiment, the enhanced DNTs are in a pharmaceutical composition, optionally with a pharmaceutically acceptable carrier.

In an embodiment, the population of enhanced DNTs exhibits an increased anti-tumor activity as compared to control DNTs that have not been contacted with the PI3Kδ inhibitor. In another embodiment, the population of enhanced DNTs exhibits an early memory status as compared to control DNTs that have not been contacted with the PI3Kδ inhibitor. In yet another embodiment, the population of enhanced DNTs exhibits a decrease in T cell exhaustion as compared to control DNTs that have not been contacted with the PI3Kδ inhibitor. In a further embodiment, the population of enhanced DNTs exhibits an increase in DNT viability as compared to control DNTs that have not been contacted with the PI3Kδ inhibitor.

The DNTs according to the present disclosure may be provided in the form of a composition. For example, in an embodiment there is provided a composition comprising the DNTs described herein, and a pharmaceutically acceptable carrier. The composition may be formulated for use or prepared for administration to a subject using a pharmaceutically acceptable carrier known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Also provided is a kit comprising DNTs and a PI3Kδ inhibitor. In an embodiment, the kit further comprises instructions for performing a method described herein, such as for producing enhanced DNTs for the treatment of cancer or for reducing the growth or proliferation of a tumor. In an embodiment, the DNTs and the PI3Kδ inhibitor are in separate containers. In another embodiment, the DNTs and the PI3Kδ inhibitor are in the same container, optionally as a composition with a pharmaceutically acceptable carrier.

Also provided is the use of the products, compositions or kits described herein for use in the treatment of cancer or in the preparation of a medicament for the treatment of cancer.

### IV. Methods and Uses of Treating Cancer and Reducing the Growth and Proliferation of a Tumor

Enhanced DNTs produced by the methods described herein have increased persistence compared to DNTs that have not been contacted with the PI3Kδ inhibitor. Accordingly, there is provided a method of treating cancer comprising administering to the subject an effective amount of enhanced DNTs. In an embodiment, the enhanced DNTs are produced by contacting the DNTs with a PI3Kδ inhibitor as described herein. In an embodiment, the PI3Kδ inhibitor is selected from the group consisting of idelalisib (Ide), PI-3065 and AZD8835. In an embodiment, the PI3Kδ inhibitor is Idelalisib (Ide).

In an embodiment, the cancer is lung cancer. In another embodiment, the cancer is leukemia. In an embodiment, the leukemia is B cell leukemia. In another embodiment, the leukemia is T cell leukemia.

In an embodiment, the leukemia is acute myeloid leukemia.

In an embodiment, the enhanced DNTs are produced from CAR-DNTs and the CAR comprises an extracellular domain specific to a suitable target antigen as described herein. In an embodiment, the CAR-DNTs are CD4-CAR-DNTs. In an embodiment, the DNTs are CD19-CAR-DNTs.

In an embodiment, the enhanced DNTs are CD4-CAR-DNTs and the cancer is T cell leukemia. In an embodiment, the enhanced DNTs are CD4-CAR-DNTs and the cancer is T cell lymphoma. In an embodiment, the enhanced DNTs are CD19-CAR-DNTs and the cancer is B cell leukemia. In an embodiment, the enhanced DNTs are CD19-CAR-DNTs and the cancer is B cell lymphoma.

Also provided is a method for reducing the growth and/or proliferation of a tumor. In an embodiment, the method comprises contacting the tumor with an effective amount of enhanced DNTs. In an embodiment, the enhanced DNTs are produced by contacting the DNTs with a PI3Kδ inhibitor as described herein. In an embodiment, the PI3Kδ inhibitor is selected from the group consisting of idelalisib (Ide), PI-3065 and AZD8835. In an embodiment, the PI3Kδ inhibitor is Idelalisib (Ide).

Also provided are uses of the enhanced DNTs, compositions, and/or kits as described herein for the treatment of cancer in a subject in need thereof. Further provided are the enhanced DNTs, compositions and/or kits for use in treating cancer in a subject in need thereof. Even further provided are uses of the enhanced DNTs, compositions, and/or kits in the manufacture of a medicament for the treatment of cancer.

Also provided are uses of the enhanced DNTs, compositions and/or kits to reduce the growth and proliferation of a tumor. Further provided are the enhanced DNTs, compositions, and/or kits described herein for use in reducing the growth and proliferation of a tumor. Even further provided are uses of the enhanced DNTs, compositions, and/or kits are in the manufacture of a medicament to reduce the growth and proliferation of a tumor.

In an embodiment, the methods and uses described herein involve the administration or use of an effective amount of enhanced DNTs.

The following non-limiting examples are illustrative of the present disclosure:

### Example 1: JQ1 does not Induce Memory Phenotype in DNTs

JQ1 was the top hit amongst 31 chemicals tested for inducing memory phenotype on conventional T cells in paper by Kagoya et al. 2016. Accordingly, the present inventors tested JQ1 for its ability to enhance DNTs therapeutic efficacy.

### Testing the Effect of JQ1 on DNTs

To investigate the effect of JQ1 on DNTs, DNTs were cultured in absence or presence of JQ1 at 0.075 μM, 0.15 μM, and 0.30 μM for 20 days. JQ1 was given to the culture on day 0, 3, 7, 10, 14, and 17. The memory status of DNTs were determined on day 14, 17, and 20 by determining the expression of T cell memory associated surface markers, CD27, CD62L, and CD45RA using flow cytometry.

### Results

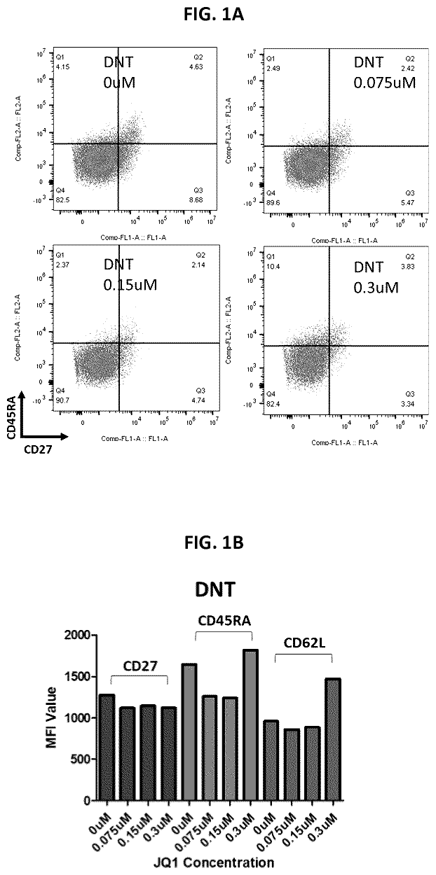

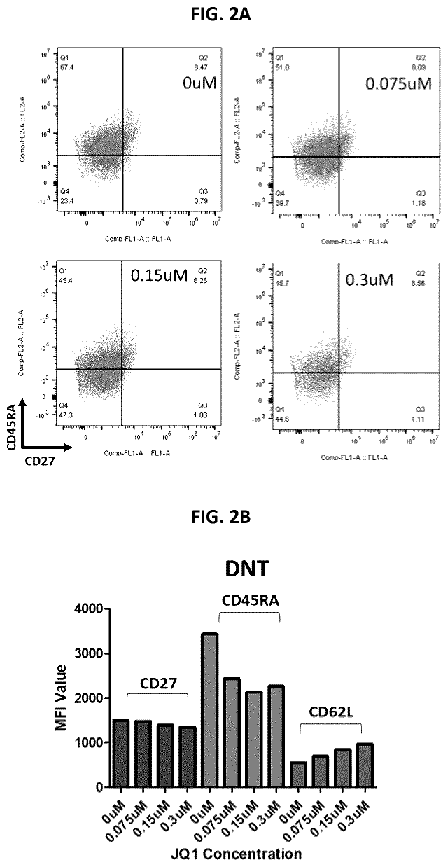

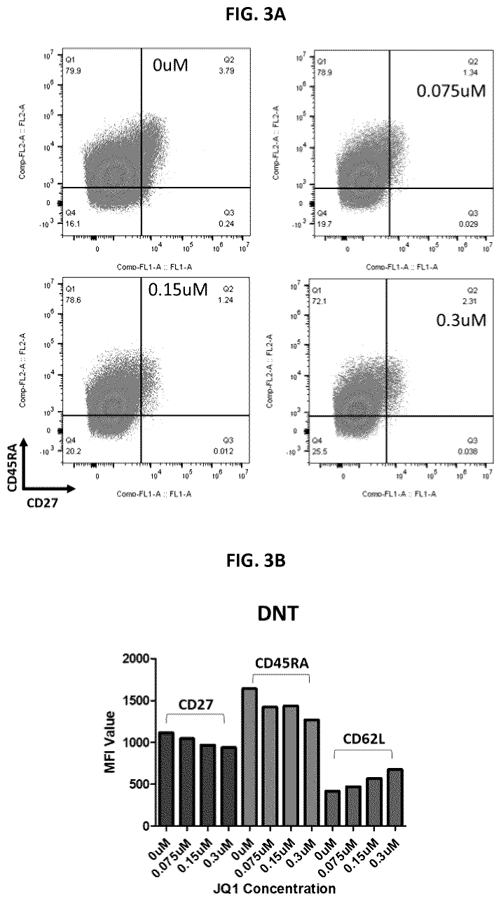

In contrast to the findings seen with conventional CD4⁺ or CD8⁺ T cells, no biologically meaningful differences on memory formation across the treatment groups on day 14 (FIGS. 1A-1B), day 17 (FIGS. 2A-2B), and day 20 (FIGS. 3A-3B) of DNT expansion was observed.

### Discussion

Despite JQ1 being the top hit for inducing memory phenotype in conventional T cells, it did not work well for DNTs.

### Example 2: Enhancing Therapeutic Efficacy of Double Negative T Cells Against Acute Myeloid Leukemia Using Idelalisib

The present inventors tested Idelalisib (Ide) to see if it was able to improve the therapeutic efficacy of DNTs.

### Materials and Methods

### Human Samples and Study Approval

Human blood from healthy donors was collected under University Health Network (UHN) Research Ethics Board (REB) approval (05-0221-T, latest approval on May 12, 2021). Frozen primary AML samples were obtained from the Leukemia Tissue Bank at Princess Margaret Cancer Centre under UHN REB (01-0573, latest approval on Mar. 25, 2021). All specimens were de-identified and given unique IDs before use. Animal studies were performed under approval by the Institutional Animal Care Committee of UHN (AUP: 741, latest approval on Jun. 4, 2021) in accordance with the Canadian Council on Animal Care Guidelines.

### Ex Vivo Expansion of DNTs and Ide Treatment

DNT was obtained and expanded as previously reported in the presence or absence of Ide (Selleckchem). Briefly, blood from healthy donor was collected in sodium heparin containing vacutainers (Becton, Dickinson and Company). Then, CD4⁺ and CD8⁺ T cells were depleted from healthy donor-derived PBMCs by incubating whole blood with RosetteSep human CD4 and CD8 depletion cocktail (StemCell Technologies) followed by density gradient centrifugation to collect CD4 and CD8 depleted buffy coats. This method successfully isolated DNT with >85% of purity and DNTs were cultured on anti-CD3 antibody-coated plates (OKT3-Biolegend) in RPMI (ThermoFisher Scientific) in the presence of 10% Fetal bovine serum (Gibco), 250IU/mL of IL-2 (Proleukin-Novartis Pharmaceuticals) and with 1 μM of Ide or equivalent amount of DMSO for three days. Soluble OKT3, IL-2 and 1 μM of Ide (Ide-DNT) or equivalent amount of DMSO (ctrl-DNT) were added to the culture every 3-4 days. DNTs were used for all functional assays between day 14 and day 20 of expansion after removal of Ide by removing supernatant after centrifugation and washing one more time with PBS.

### Antibodies and Flow Cytometry

The following antibodies were used in this study: CD4-FITC or -APCcy7, CD56-PE, CD3-PEcy7 or -Alexa Flour700, CD8-APC or -APCcy7, CD45RA-FITC, CD95-APC, CD62L-PEcy7, CTLA4-PE, LAG3-PEcy7, TIM3-APC, CD45-FITC or -APCcy7, CD34-PE, CD33-APC, AnnexinV-Pacific Blue or -FITC, 7AAD, IFNy-PE, TNFα-APC and DAPI. All listed anti-human antibodies were purchased from BioLegend. Data were obtained using Attune NXT flow Cytometer (ThermoFisher) and analyzed with Flowjo software (Tree Star, Inc.).

### Patient Derived Xenograft Models

6-12 weeks old NOD.Cg-Prkdcscid II2rgtm1Wjl/SzJ (NSG) mice were purchased from Jackson Laboratories and housed in the Animal Resource Centre at UHN. Mice were sublethally irradiated (2.5 Gy) one day prior to intravenous injection of 2-3×10⁶ AML blasts obtained from patients. On day 17 and day 20, mice were injected with PBS or 20×10⁶ Ide-DNT or ctrl-DNTs with 10,000 IU of IL-2 intravenously and followed by weekly 10,000 IU of IL-2 intraperitoneal injections. Mice body weight was measured twice a week. On day 24-100 mice were euthanized and bone marrow (BM), spleen, liver and lung were harvested. For liver and lung, small tissue sections were fixed in 10% formalin and sent for hematoxylin and eosin (H&E) staining. The rest of tissues were digested with collagenase D at 37° C. for 30 minutes, filtered through 40 μm cell strainer, and underwent density gradient centrifugation at 1200 g for 20 minutes. The buffy coats were collected to check for the frequency and number of DNTs and AML using flow cytometry, as described previously. To longitudinally monitor the DNT level in mice periphery, 75-100 μL of blood was collected from lateral saphenous vein weekly, and red blood cells were lysed using Ammonium Chloride (StemCell Technologies). Subsequently, DNT and AML levels were assessed by flow cytometry.

### Cytokine Production Assay

Day 14-20 ex vivo expanded DNTs were cultured in medium for 4 hours with anti-CD3/anti-CD28 dynabeads (Gibco) and ×1 protein transport inhibitor cocktail (eBioscience) following the manufacturer's instructions. Cells were harvested after stimulation, fixed and permeabilized with fixation buffer (BioLegend) and perm wash buffer (BioLegend), respectively. Fixed and permeabilized cells were intracellularly stained using anti-human cytokine antibodies as listed above.

### In Vitro Cytotoxicity Assay

DNTs were co-cultured with AML cells at 0.25:1 to 4:1 effector-to-target ratios for 2 hours or 20 hours in 96 well plates and apoptosis of targeted cells was measured using flow cytometry. Specific killing of targets was calculated by

$$\frac{\begin{matrix}\% AnnexinV of AML with DNT - \\ \% AnnexinV of AMLwithout DNT\end{matrix}}{100 - \% AnnexinV of AML without DNT}$$

and viability of effector cells was calculated by 100-% AnnexinV⁺ in cultures of DNTs with AML.

### Real-Time PCR

RNA was isolated from day 14-20 ex vivo expanded DNTs using RNeasy Mini Kits (Qiagen) following the manufacturer's protocol. cDNA was synthesized from the isolated mRNA using QuantiTect Reverse Transcription Kit (Qiagen) according to the manufacturer's protocol. Real-time PCR was performed on the LightCycler 480 Instrument (Roche Life Science) using the LightCycler 480 SYBR Green I Master (Roche). Data were analyzed using LightCycler 480 Software version 1.5. Primers that were used for all qPCR were obtained from Integrated DNA Technologies.

### Statistical Analysis

All statistical analyses were performed using GraphPad Prism 5. The data were analyzed by two-tailed student's t test, linear regression tests and two-way ANOVA where statistical significance was set as *P<05, **P<. 01, ***P<. 001, and ****P<0001. Graphs are presented with meant SEM.

### Results:

### DNT Persistence and Anti-Leukemic Activity in Leukemia Bearing Hosts

A number of studies highlights the importance of infused cell persistence for favourable clinical outcome of ACT [19-25]. Previously, it has been demonstrated that adoptively transferred human DNTs could persist in naïve NSG mice for 2-3 weeks [17,26]. However, DNT engraftment in leukemia bearing host may differ from those seen in naive mice as AML can hamper immune cell survival and anti-leukemic activity via immunosuppressive function or promote their function and persistence by providing stimulatory signals for T cells to proliferate [27]. Therefore, the persistence of DNTs in leukemia-bearing hosts was investigated for better resemblance of clinical setting.

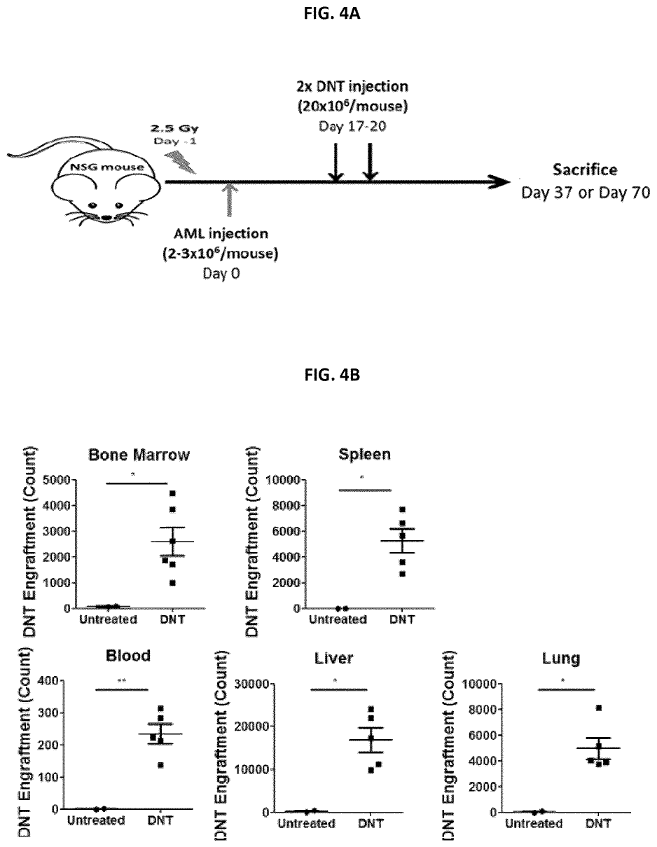

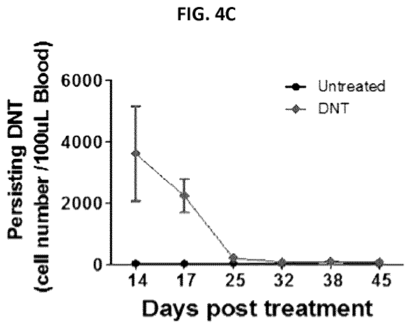

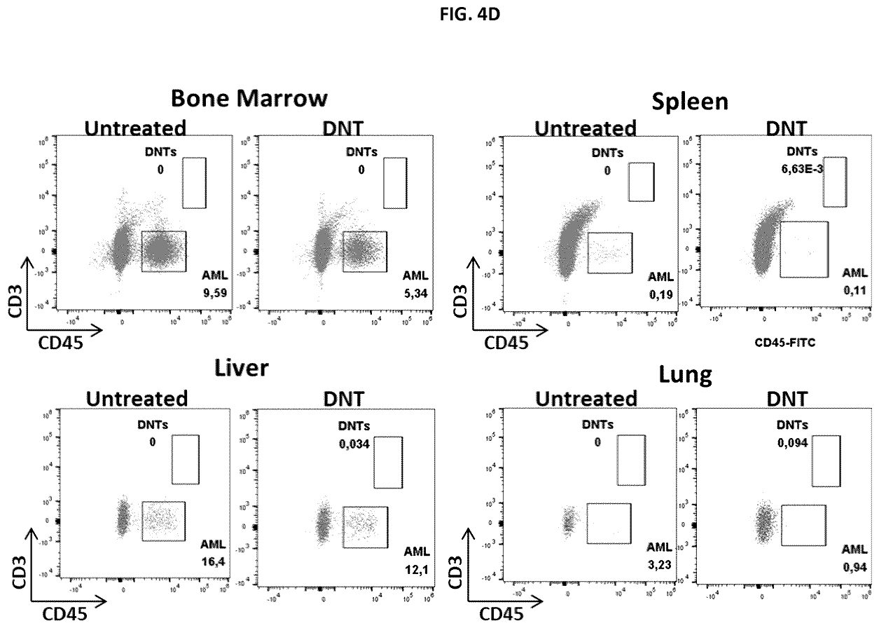

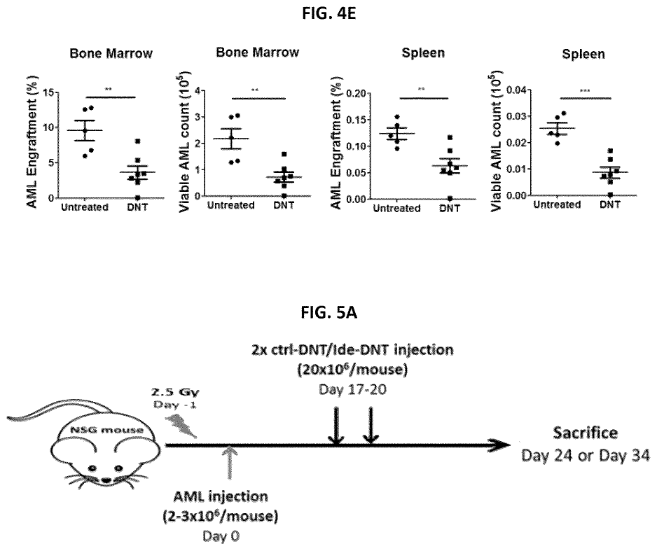

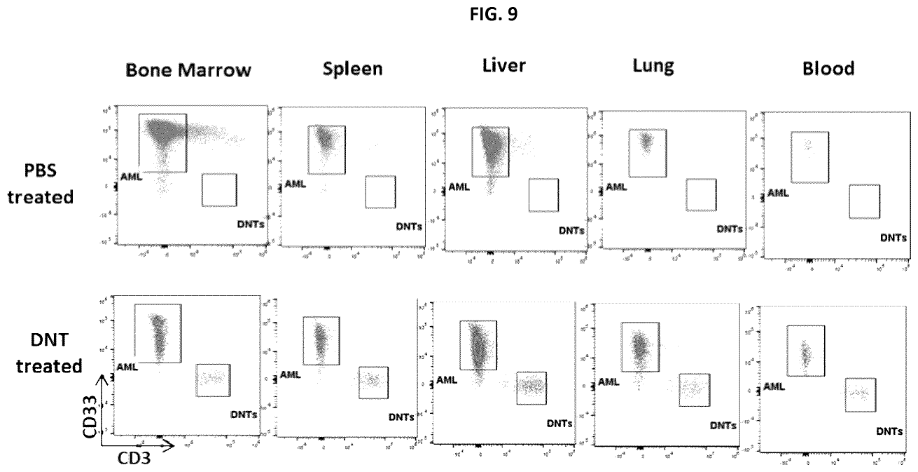

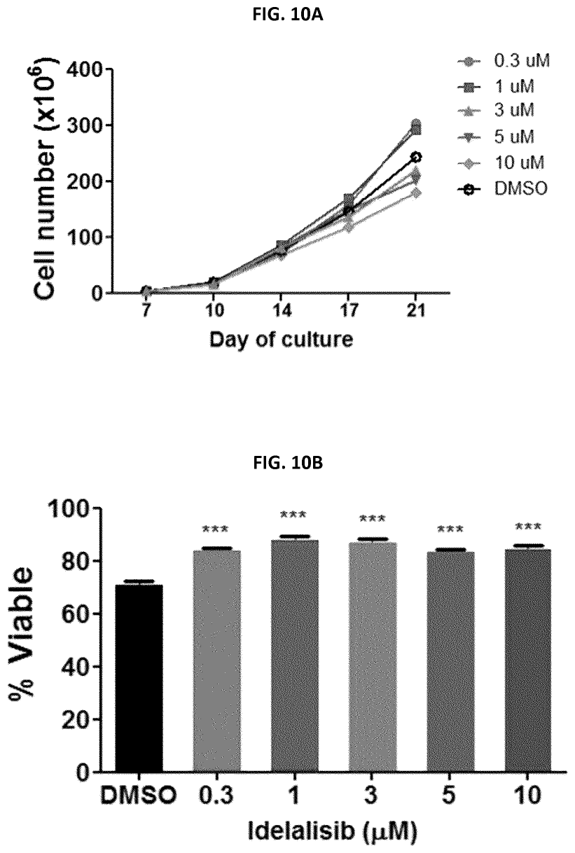

To test this, sublethally irradiated NSG mice were inoculated with primary AML patient blasts followed by infusion of two doses of ex vivo expanded DNTs or PBS. Subsequently, DNT levels were monitored in various tissues (FIG. 4A). DNTs were detectable in blood, spleen, BM, liver, and lung on day 37 which was 17 days post—the second DNT treatment (FIGS. 4B and 9). Further, DNTs were detectable in peripheral blood up to 25 days post DNT treatment but were no longer detectable after 32 days post DNT treatment which were day 45 and day 52, respectively (FIG. 4C). By day 70, 50 days post-treatment, no DNTs were detectable in the BM with negligible levels of DNTs found in the spleen (0.016±0.004%), liver (0.122±0.054%) and lung (0.23±0.0522%) (FIG. 4D). DNTs significantly reduced the frequency and total number of AML cells in the BM and spleen compared to the PBS-treated group (FIGS. 4D and 4E). However, significant levels of residual AML cells were detected with very little to no DNTs persisting (FIGS. 4D and 4E). Given that residual AML cells detected after clearance of DNTs can significantly lessen the significance of DNT-mediated anti-leukemic activity, ways to improve DNT persistence in vivo was investigated.

### Ide Significantly Enhances Anti-Leukemic Function of DNTs in Patient-Derived Xenograft Model

When T cells are activated through T cell receptor (TCR), co-stimulatory molecules, and cytokine receptor engagements, PI3K pathway is activated predominantly through PI3Kδ. Active PI3K pathway then initiates T cell activation and differentiation that generate effector T cells with robust effector function but with low persistence [28,29]. To improve DNT persistence and its long term anti-leukemic activity in vivo, the impact of treating DNTs with an FDA-approved inhibitor against PI3Kδ, Ide, was investigated.

First, DNTs were isolated and expanded as previously described in the presence of increasing concentrations of Ide. Compared to the vehicle control, improved cell yield was observed at the end of expansion in the presence of 0.1-1.0 μM Ide, while DNT expansion was compromised from 3.0-10.0 μM of Ide (FIG. 10A). In addition, 1 μM Ide resulted in the highest DNT viability at the end of cell expansion cultures (FIG. 10B). Based on these results, 1 μM Ide was used to treat DNTs for the rest of the study.

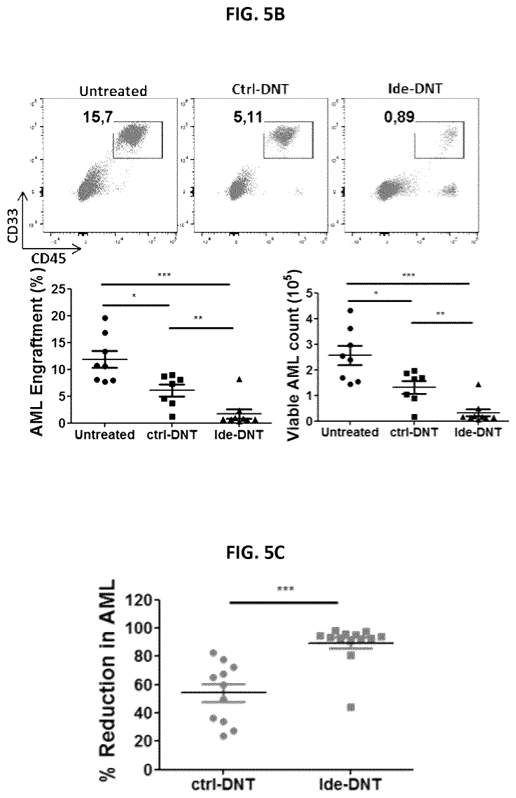

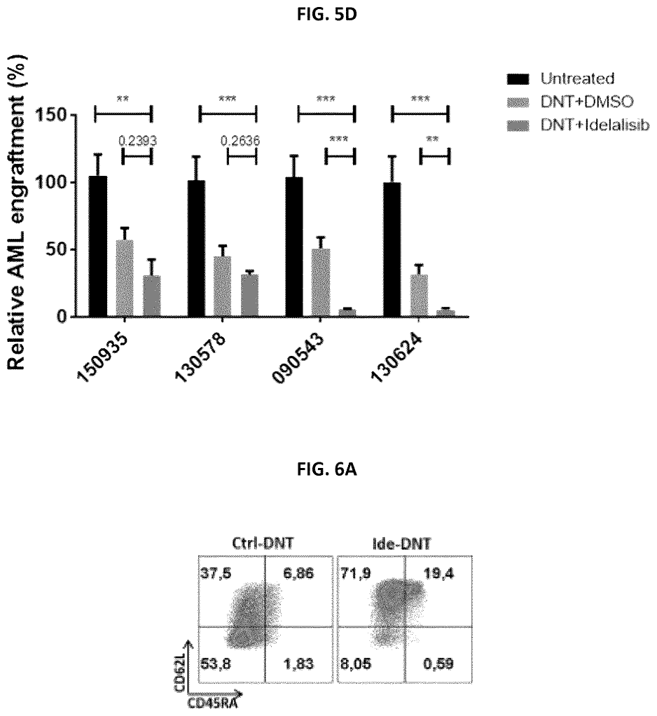

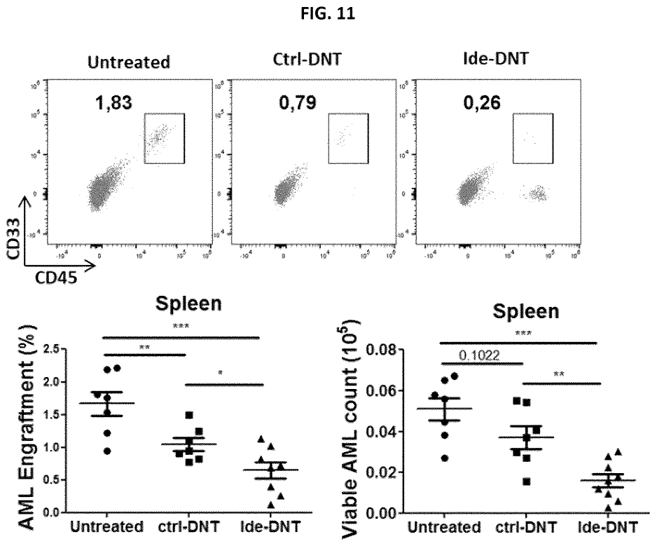

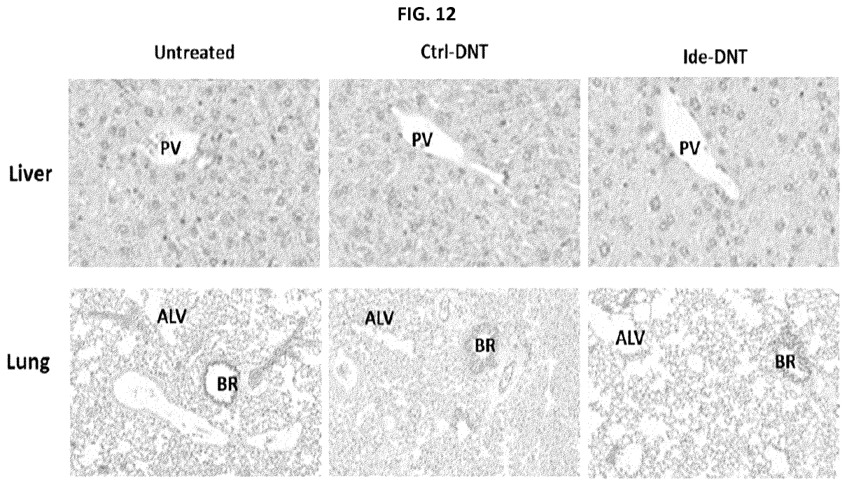

Next, the anti-leukemic activity of Ide-DNTs was investigated using previously established AML patient-derived xenograft (PDX) models [13]. Briefly, NSG mice engrafted with primary AML samples were treated on day 17 and day 20 with PBS, DNTs expanded in the presence of Ide or in the presence of vehicle control. Subsequently, AML engraftment was assessed on day 24 or day 34, 4 days or 14 days post-treatment, respectively (FIG. 5A). Notably, significantly lower AML levels were observed in Ide-DNT treated mice compared to untreated and ctrl-DNT treated groups on day 34 (FIGS. 5B and 11), where AML counts in BM were 16.38±3.53 and 9.6±2.07 fold lower in Ide-DNT group relative to untreated and ctrl-DNT treated groups, respectively. Summarized results from PDX experiments done using primary AML samples from two different patients show 89.44±3.95% reduction in AML engraftment with Ide-DNT treatment versus 54.27±6.35% reduction with ctrl-DNTs (FIG. 5C). Interestingly, when AML engraftment level was assessed 4 days post DNT infusion, Ide-DNT group showed comparable AML engraftment level as ctrl-DNT group (FIG. 5D). In contrast, superior anti-leukemic activity of Ide-DNT relative to ctrl-DNT was detected 14 days post DNT infusion (FIG. 5D). This suggests that the superior anti-leukemic activity of Ide-DNT may be attributable to prolongation of its activity instead of improved potency. Importantly, despite the improved cytotoxicity towards leukemic blasts, off-tumor toxicity of DNTs remained undetectable with or without Ide-treatment (FIG. 12).

Ide Promotes an Early Memory Subset of DNTs that have Better Proliferative Capacity

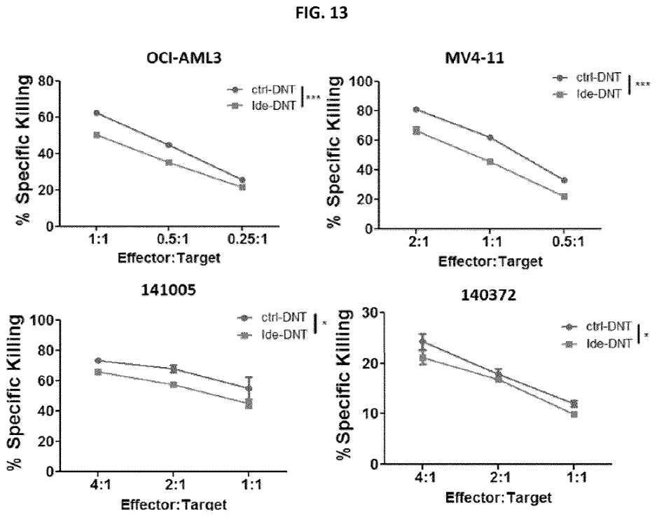

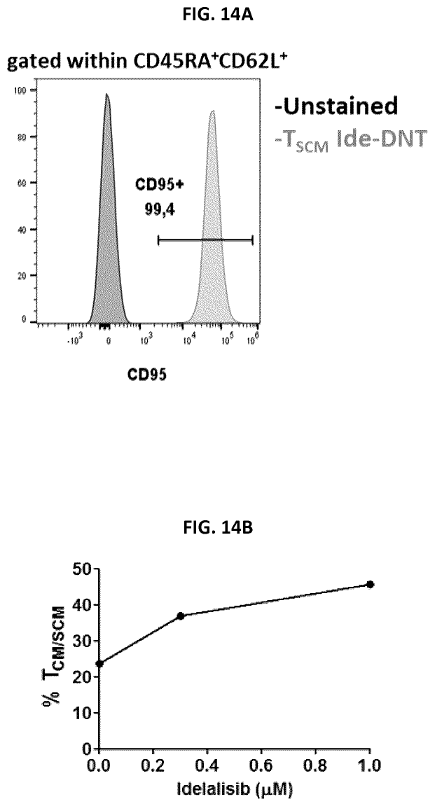

To determine the mechanisms by which Ide enhances DNT function, the effect of Ide on the cytotoxic activity of DNTs in vitro was first assessed. Ide-DNT mediated slightly, but statistically significantly, reduced cytotoxicity against AML cell lines including OCI-AML3 and MV4-11 and primary AML blasts than ctrl-DNTs in vitro (FIG. 13). The reduced 2-hour cytotoxicity (FIG. 13), along with the comparable leukemia burden seen in Ide-DNT and ctrl-DNT groups 4 days post DNT infusion (FIG. 5D), supported the idea that the superior in vivo activity of Ide-DNTs is not due to elevated robustness of DNT-mediated cytotoxicity against AML.

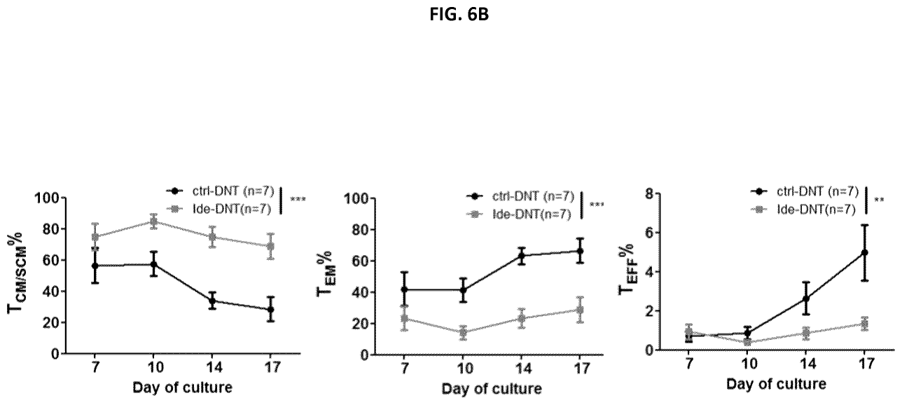

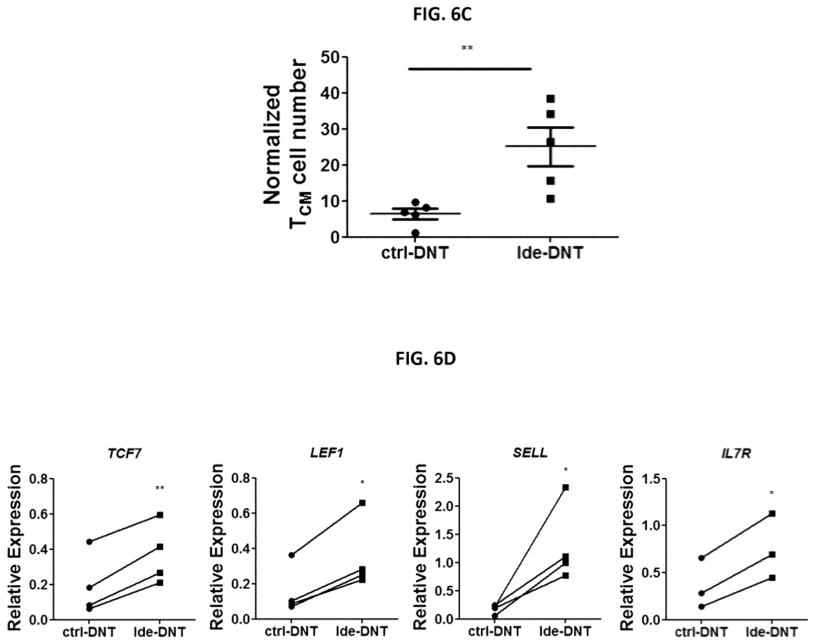

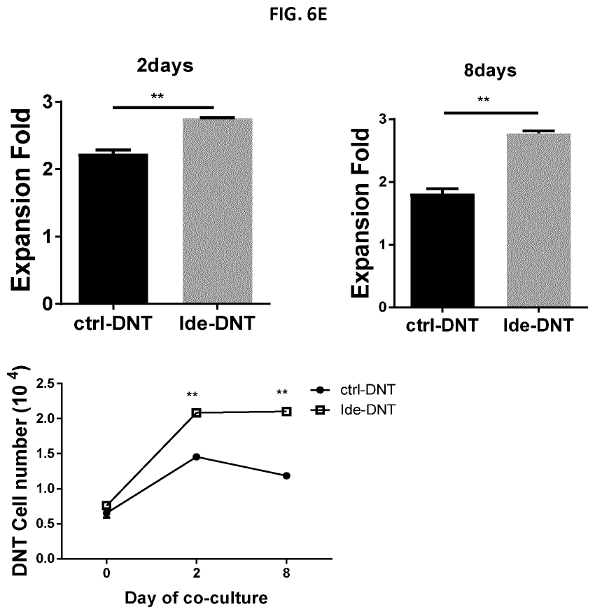

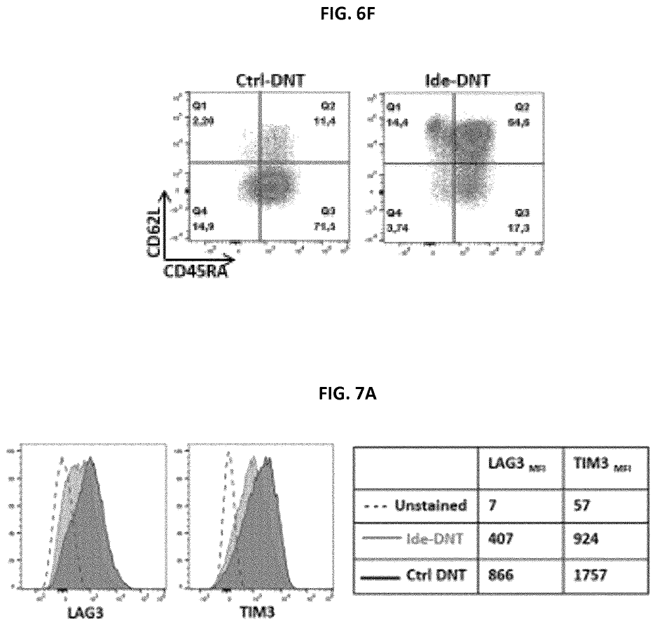

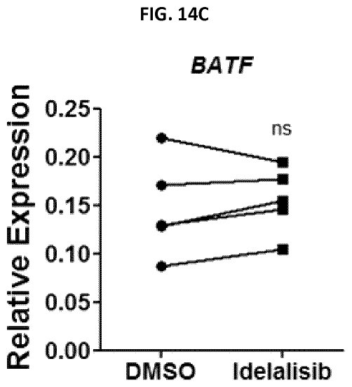

Given that the significant enhancement of anti-leukemic activity of Ide-DNT was observed 14 days, but not 4 days after treatment, the impact of Ide on DNT memory status was investigated. Early memory phenotype of infused cells is an important dictator for immune cell persistence that is linked to a better clinical outcome [30,31]. Notably, the majority of expanded Ide-DNTs exhibited central memory (T_{CM}; CD45RA⁻CD62L⁺) and stem cell memory (T_{SCM}; CD45RA⁺CD62L⁺CD95⁺) phenotypes, while effector memory (T_{EM}; CD45RA-CD62L⁻) DNTs were the dominant subset in ctrl-DNTs (FIGS. 6A and 14A). A significantly higher proportion of early memory subsets, T_{CM}/T_{SCM}, and lower frequency of T_{EM} and T_{EFF} populations were observed in Ide-DNTs relative to ctrl-DNTs throughout DNT expansion with more notable differences at a later stage of expansion, between day 14 and day 17 (FIG. 6B). As a result, there was a 3.87±0.82-fold higher T_{CM}/T_{SCM} cell number obtained in the presence of Ide at the end of expansion compared to ctrl-DNTs (FIG. 6C). Further, these changes towards early memory phenotype were observed from Ide-DNTs in a dose-dependent manner (FIG. 14B). Consistent with this, stem cell memory-associated transcription factor genes, TCF7 and LEF1, were expressed at a higher level by Ide-DNTs than ctrl-DNTs between day 14 and day 17 of culture, as well as memory associated genes, SELL and IL7R (FIG. 6D). However, insignificant change was observed in BATF expression, which encodes a transcription factor important for effector T cell differentiation (FIG. 14C).

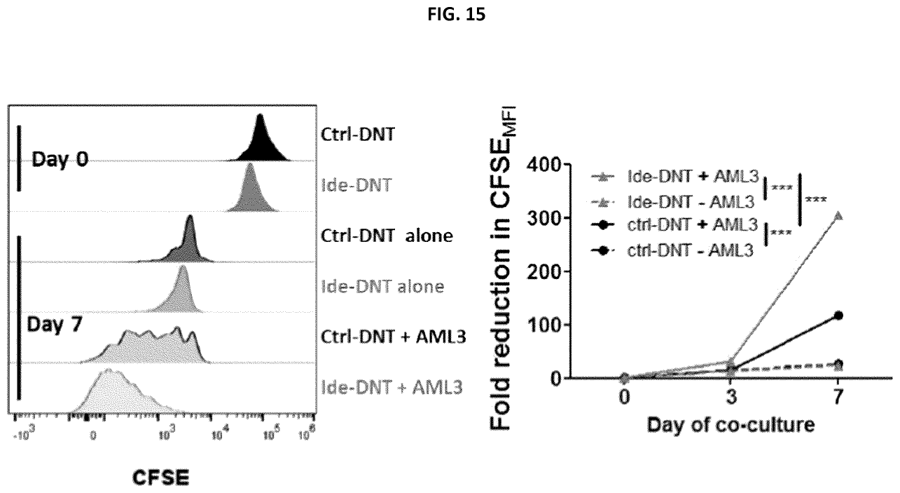

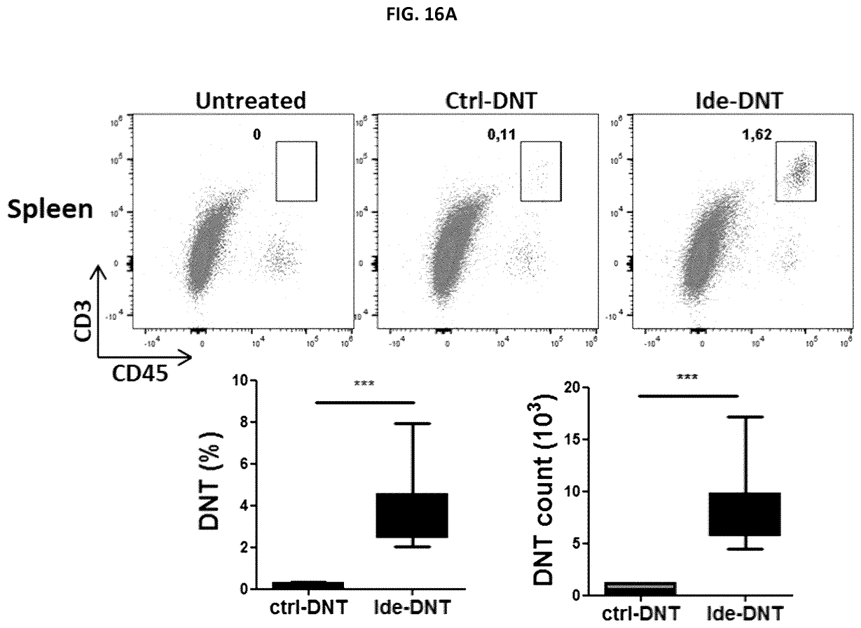

In addition to genetic and molecular markers, a key biological feature of memory T cells is their ability to persist and proliferate after encountering their targets [33]. Hence, the proliferative capacity and viable cell counts for Ide-DNTs and ctrl-DNTs during co-culture with an AML cell line, OCI-AML3, were monitored for 8 days. A significantly higher degree of proliferation (FIG. 15) and number (FIG. 6E) of viable DNTs was observed when Ide-DNTs were co-cultured with AML cells than ctrl-DNT. Importantly, their early memory phenotype was maintained for at least 8 days post co-culture with AML targets, while control DNTs presented a terminally differentiated effector phenotype (FIG. 6F). Collectively, these observations demonstrate that Ide promotes an early memory status of DNTs that may contribute to the superior in vivo leukemia control of Ide-DNT.

### Ide Improves Fitness of DNTs by Inhibiting Exhaustion and Enhancing Viability

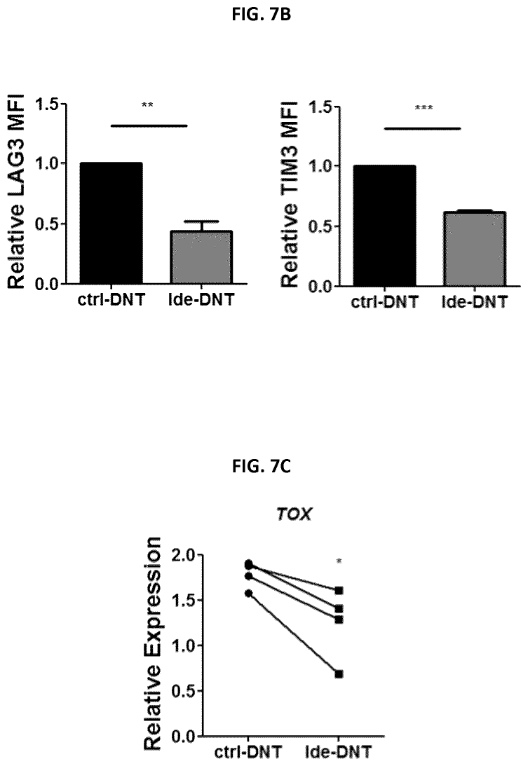

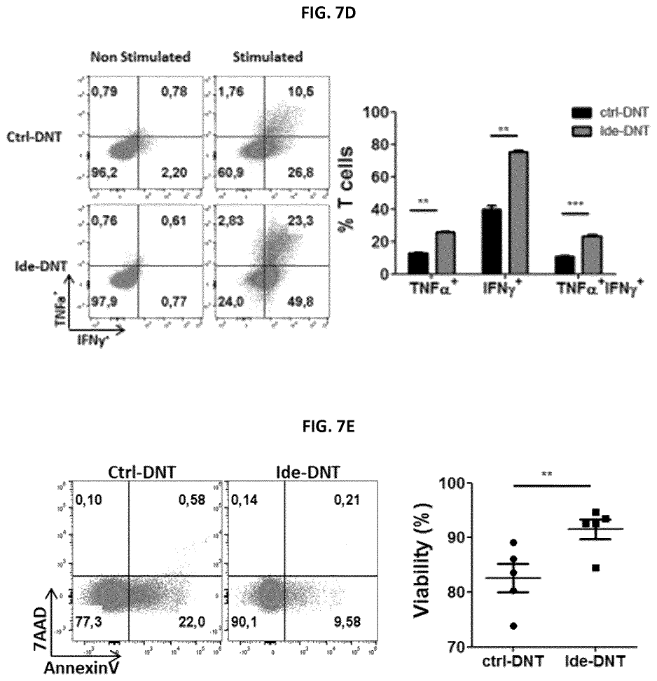

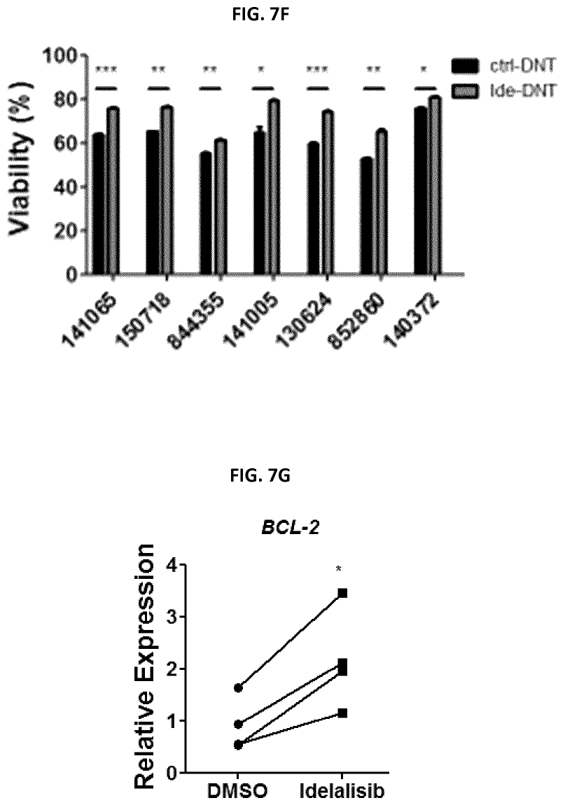

Next, the effect of Ide on DNT exhaustion was studied as T cell exhaustion can significantly impair in vivo anti-tumoral activity [30,34]. Ide-treatment reduced the expression of inhibitory receptors, Lag3 and Tim3, on DNTs compared to the ctrl-DNTs (FIGS. 7A and 7B). Furthermore, Ide-DNTs exhibited significantly lower expression of TOX, a transcription factor gene known as the central regulator of T cell exhaustion (FIG. 7C). Functionally, Ide-DNTs mediated superior single (IFNy⁺ or TNFα⁺) and multi-cytokine (IFNy⁺ and TNFα⁺) production after stimulation, further supporting the less exhausted state than control DNTs (FIG. 7D).

Further, Ide significantly improved the viability of DNTs from 5 healthy donors with reduced apoptotic DNTs after expansion (FIG. 7E). Moreover, Ide-DNTs remained more viable after co-culture with primary AML blasts from 7 patients (FIG. 7F). In agreement with this, higher expression of anti-apoptotic gene, BCL-2 was detected from Ide-DNTs compared to ctrl-DNTs (FIG. 7G). Collectively, these results demonstrate that Ide prevents exhaustion and improves viability of DNTs, resulting in DNTs with a better fitness at the end of expansion.

### Ide Prolongs Persistence of DNTs and Promotes a Durable Anti-Leukemic Effect

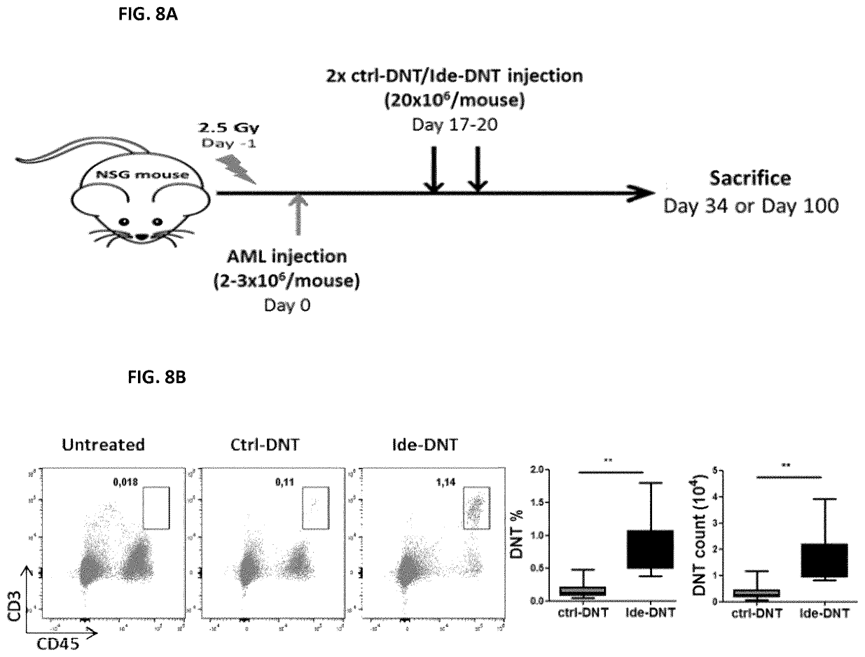

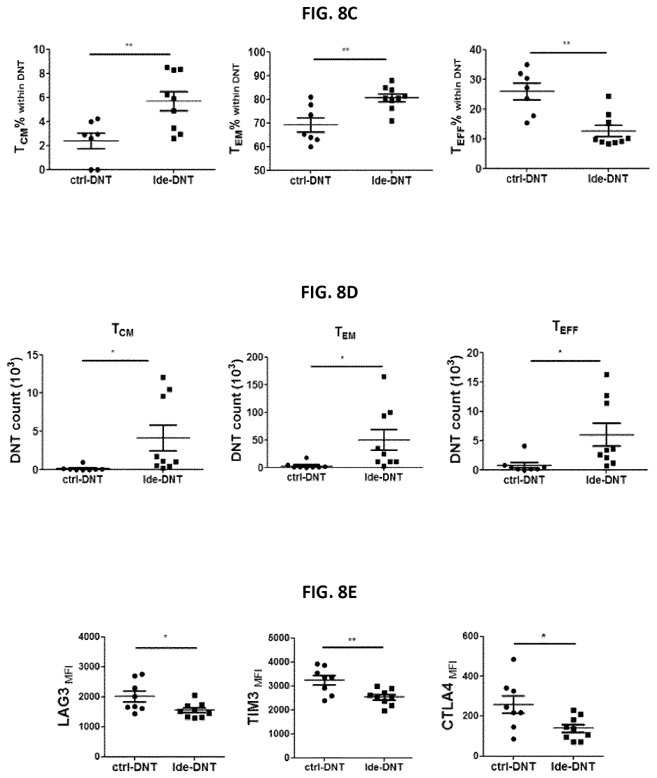

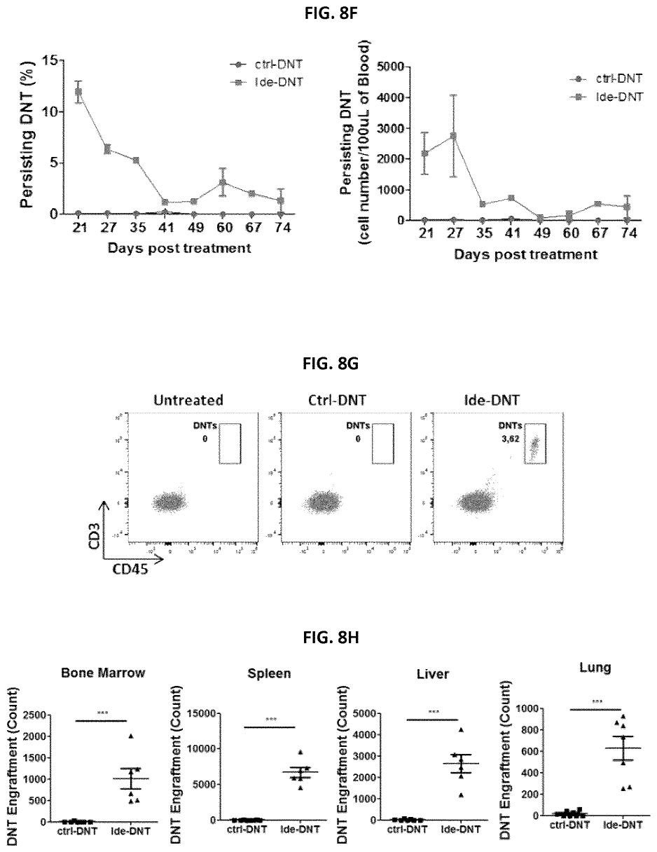

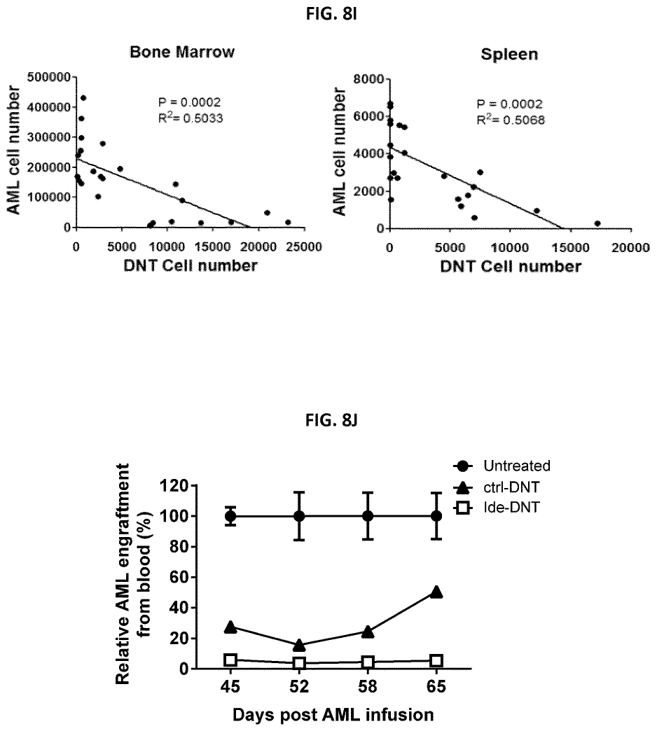

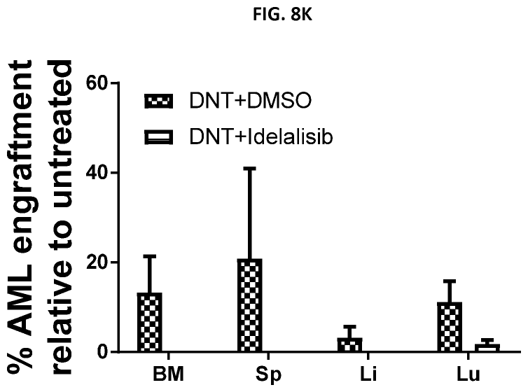

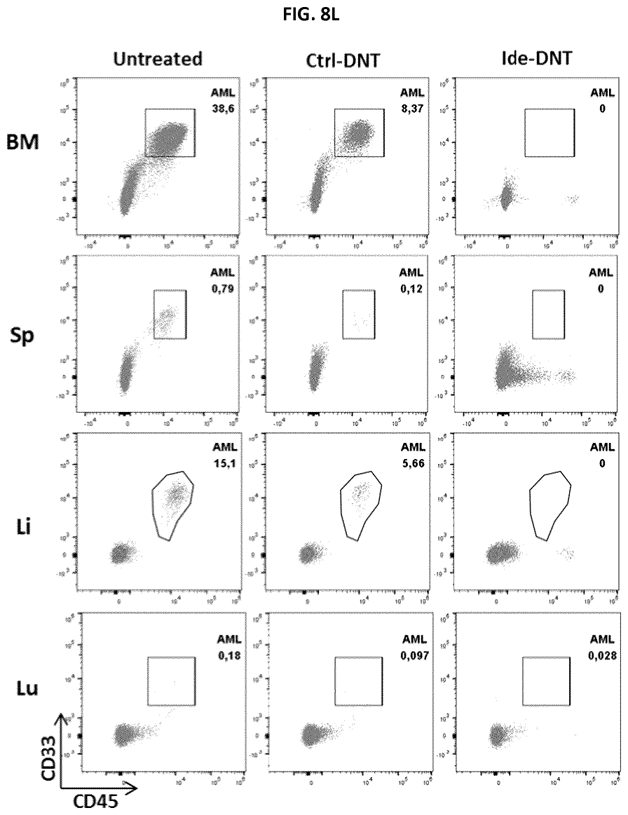

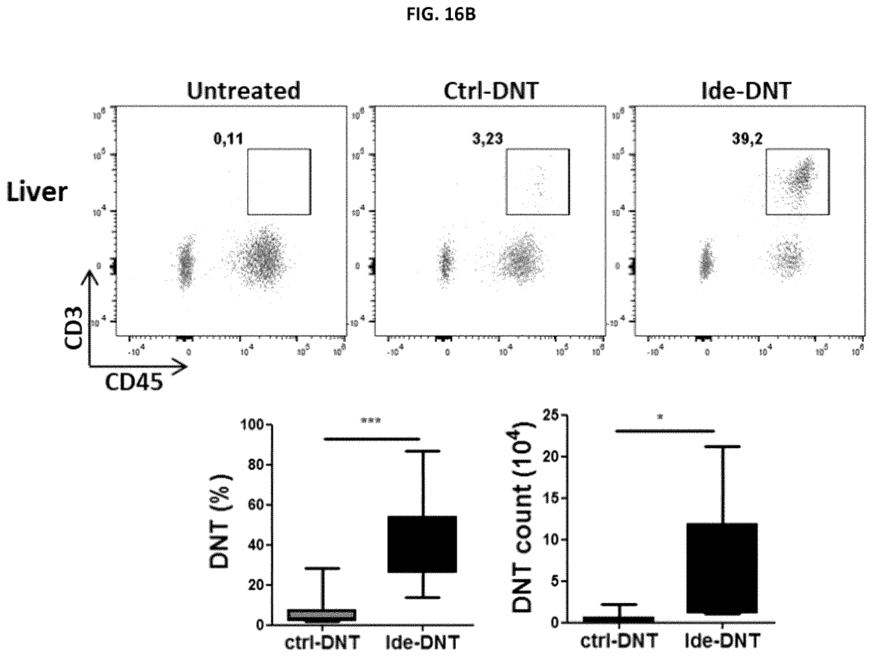

Given that Ide skews DNTs towards various characteristics associated with improved persistence of immune cells, such as early memory status, improved proliferative capacity, and reduced exhaustion, the distribution and persistence of Ide-DNT and ctrl-DNT was monitored using the PDX model described above (FIG. 8A). Compared to the ctrl-DNT group, not only did Ide-DNTs show improved persistence in lymphoid organs like BM (FIG. 8B) and spleen (FIG. 16A), but a significantly increased number of DNTs in non-lymphoid organs like liver (FIG. 16B) on day 34, which was 14 days post DNT infusion, was also observed. Furthermore, Ide-DNTs detected in xenografted mice were significantly less differentiated than ctrl-DNTs with a higher proportion of T_{CM} and T_{EM} subsets (FIG. 8C). In contrast, a higher frequency of persisting ctrl-DNTs was observed in the terminally differentiated effector subset (T_{EFF}) (FIG. 8C). Moreover, a 27.46±11.13 and 7.57±2.45-fold increase in the total number of DNTCM and DNTEM were observed in the Ide-DNT group relative to the ctrl-DNT group (FIG. 8D). Interestingly, while DNTEFF frequency was lower, the total number of DNTEFF was 15.85±5.91 fold higher in the Ide-DNT group compared to ctrl-DNT group (FIGS. 8C and 8D). Consistent with the in vitro observation, Ide-DNTs were less exhausted than ctrl-DNTs (FIG. 8E).

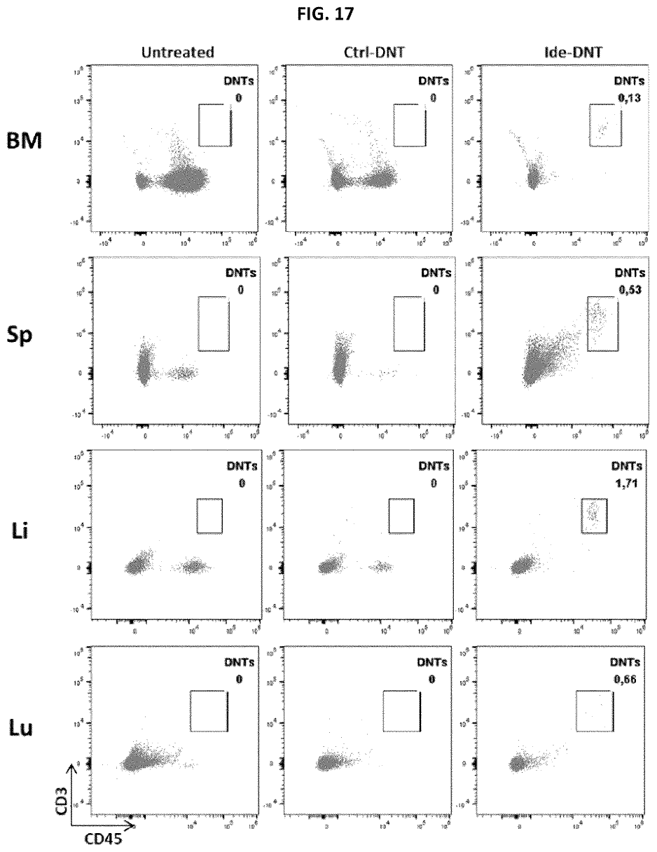

Next, the long-term persistence of DNTs was studied by monitoring the DNT levels in blood up to 74 days post DNT infusion (FIG. 8F). A significantly higher number and percentage of DNTs were detected in blood from day 21 to day 74 post DNT infusion in Ide-DNT treated mice compared to ctrl-DNTs treated ones (FIGS. 8F and 8G). In addition, DNTs were detectable in the Ide-DNT group in various organs including BM, spleen, liver, and lung at least up to 80 days post-treatment which was on day 100, but not in the ctrl-DNT group (FIGS. 8H and 17). These results collectively support the significantly improved engraftment and persistence of DNTs when expanded in the presence of Ide.

Lastly, whether the prolonged persistence of Ide-DNTs led to not only superior but also more durable anti-leukemic activity compared to ctrl-DNTs was studied. Notably, the significant inverse correlations were observed between the number of AML cells and DNTs in BM and spleen on day 34, where higher DNT counts correlated with lower AML engraftment, demonstrating the importance of DNT persistence in leukemic clearance (FIG. 8I). Similarly, AML cells were undetectable in the peripheral blood of Ide-DNT treated mice from day 45 while detectable levels of peripheral AML blasts of ctrl-DNT treated mice were found on day 45, albeit at a lower level than the untreated group (FIG. 8J). Further, while the peripheral AML counts of Ide-DNT treated mice remained undetectable up to day 65, the peripheral AML counts of ctrl-DNT treated mice increased from day 52 to day 65 (FIG. 8J). In another PDX experiment conducted with patient AML blasts that migrate systemically, Ide-DNT eradicated AML cells from both lymphoid (BM and spleen) and non-lymphoid (liver and lung) organs and kept mice in a leukemia-free state for at least 100 days (FIGS. 8K and 8L). In contrast, AML cells were detected from all organs of ctrl-DNT treated mice although at lower levels than the untreated group (FIGS. 8K and 8L). Collectively, these results support the notion that Ide enhances the potency and durability of DNT-mediated leukemia clearance by enhancing the overall fitness and persistence of expanded DNTs.

### Discussion

Previously, the therapeutic potential of DNTs as an off-the-shelf therapy for AML patients was demonstrated [12,13,17]. A recently completed phase I/IIa clinical trial assessed the activity of allogeneic DNTs in AML patients with relapsing disease after allo-HSCT and supported the feasibility, safety, and potential efficacy of allogeneic DNTs for this patient population associated with very poor prognosis [18]. While the above study monitored the total DNT levels in the treated patients, the level and persistence of donor-derived DNTs were not examined. Recent studies reported that ex vivo expanded human DNTs can persist for around 14-21 days in naïve mice [17,26]. However, DNT persistence in leukemic bearing hosts, which provides more clinically relevant settings, was never studied. In this study, the persistence of ex vivo expanded DNTs in leukemia-bearing hosts was investigated and it was demonstrated that expanding DNTs in the presence of Ide can significantly improve their persistence, therapeutic efficacy and durability of response against AML in PDX models.

Consistent with previous findings, ctrl-DNTs mediated significant anti-leukemic effect in PDX models. The ctrl-DNTs were detectable in blood up to 25-32 days post DNT infusion in leukemia-bearing hosts and in spleen, liver and lung up to 50 days post DNT infusion, although the detectable level was very low. The prolonged persistence in leukemia-bearing hosts relative to naïve hosts suggests that the leukemic cells may provide stimulatory signals to DNTs to promote their persistence in vivo. However, the AML level increased starting on day 52 at which time no significant number of DNTs was detected in the peripheral blood of ctrl-DNT treated mice, suggesting the lack of persistence of ctrl-DNTs may be a limiting factor for a durable anti-leukemic response.

PI3Kδ plays a central role in initiating clonal expansion and differentiation of activated T cells to develop effective anti-tumour activity [29,35,36]. However, the fine balance of activation signal is important to minimize terminal differentiation and exhaustion of anti-leukemic T cells [37-39]. Given the strong correlation between the activation and exhaustion status of T cells and their in vivo persistence, a PI3Kδ inhibitor, Ide, was used to overcome the hurdle of limited in vivo persistence and durability of response of DNTs. Notably, Ide slightly reduced potency of DNTs against AML blasts in vitro on a per cell basis, and anti-leukemic activity in ctrl-DNT and Ide-DNT groups were comparable when assessed 4 days post-treatment. However, significant improvement of anti-leukemic activity was observed in Ide-treated DNTs compared to ctrl-DNTs when assessed at least 14 days post-treatment. Collectively, these results suggest that the superior anti-leukemic responses seen with Ide-DNT are more likely due to improved persistence of DNTs rather than enhancing their cytotoxicity.

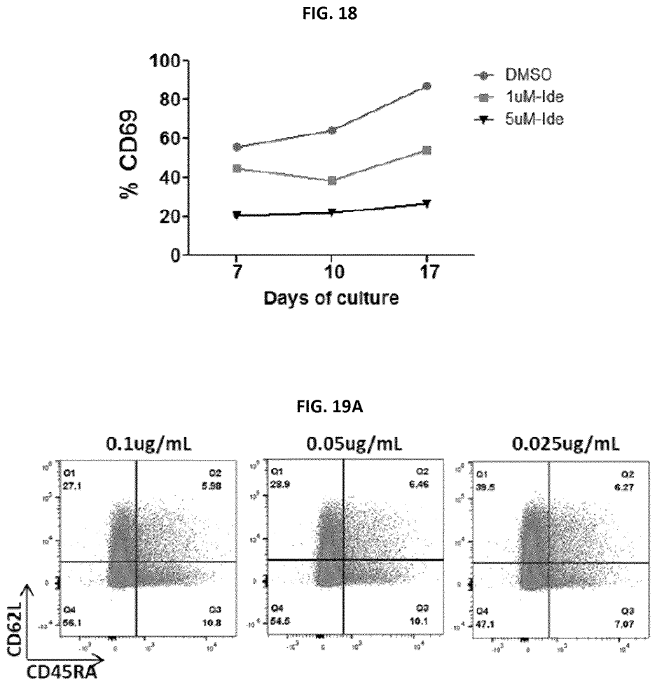

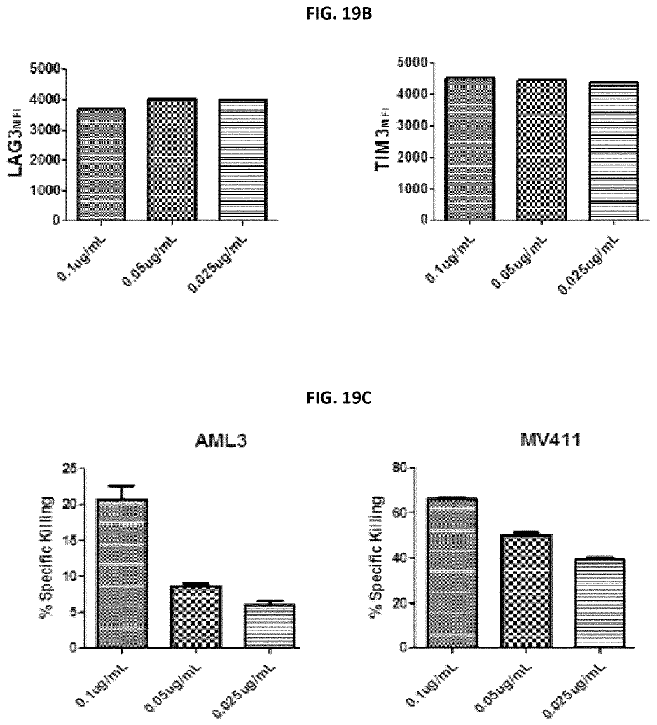

Mechanistically, Ide skewed DNTs towards T_{CM} and T_{SCM} subsets with increased expression of stem cell memory-associated genes in Ide-DNTs versus ctrl-DNTs. Blocking of PI3Kδ with Ide significantly dampened DNT activation signal as shown from reduced levels of CD69 expression in a dose-dependent manner (FIG. 18). However, lowering the amount of anti-CD3 antibody given during expansion did not recapitulate the memory and exhaustion status seen with Ide (FIGS. 19A and 19B), although it significantly reduced the cytotoxicity function against AML (FIG. 19C). This finding suggests that Ide-mediated enhancement of DNT-persistence is not simply due to the dampening of TCR signalling. In addition, it should be noted that Ide concentrations higher than 1 μM significantly compromised the expansion rate of DNTs (FIG. 10A). This suggests rather than disposing PI3Kδ signalling, a fine balance of the PI3K activation pathway is required to warrant optimal DNT expansion.

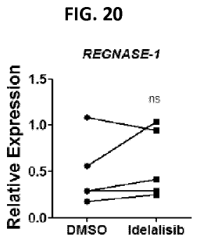

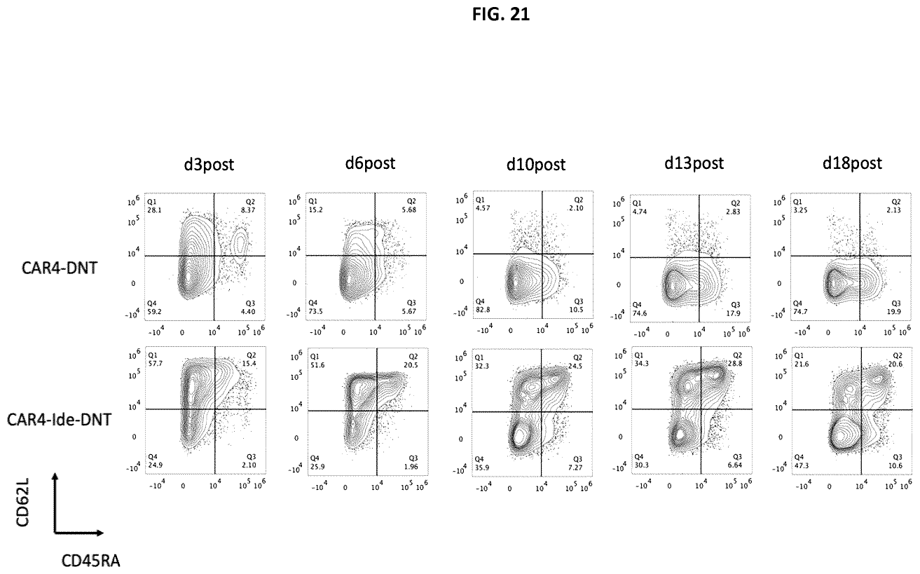

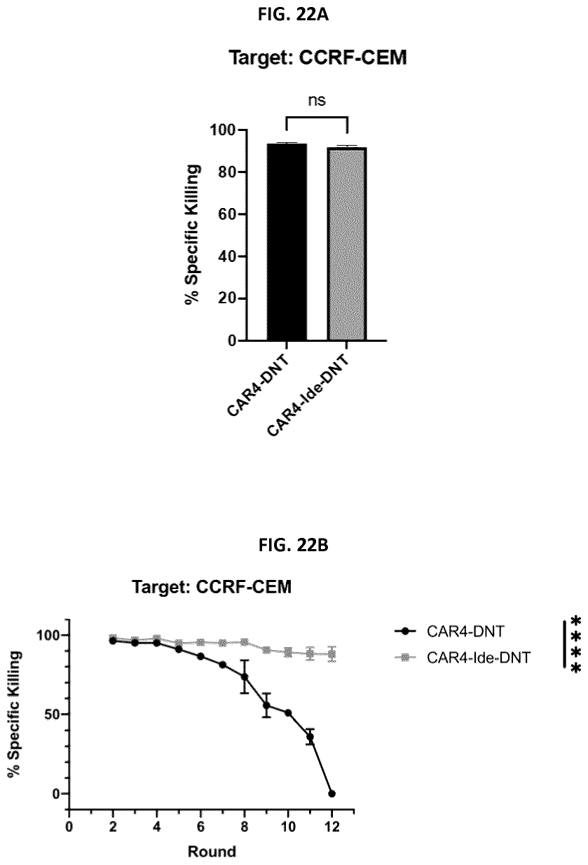

T cell exhaustion affects proliferative capacity as well as cytotoxicity activity of T cells against tumours, resulting in poor persistence and overall outcomes [30,34]. PI3Kδ blockade reduced exhaustion level of DNTs, leading to superior proliferative capacity and ability to produce effector cytokines (IFNγ and TNFα) upon stimulation. Moreover, lower expression of transcription factor TOX was detected. However, further investigation is needed to determine if the lower TOX expression seen in Ide-DNT is responsible for the less exhausted state of DNTs. A recent study reported that Regnase-1, a ribonuclease that regulates immune cell activation, as a crucial negative regulator for programming high Lef1 and Tcf7 but low Tox expressing CD8⁺ T cells by suppressing BATF [40,41]. Although lower expression of TOX and high expression of LEF1 and TCF7 was observed in Ide-DNTs, comparable levels of REGNASE-1 and BATF were observed between Ide-DNTs and ctrl-DNTs, suggesting that the Ide-mediated effect is Reganse-1 independent (FIGS. 14C and 20).

Ide is an FDA-approved drug for treatment of lymphoma including chronic lymphocytic leukemia, follicular B-cell non-Hodgkin lymphoma and small lymphocytic leukemia [42,43]. However, severe adverse events associated with Ide-treatment have been reported including autoimmune toxicities, infection, diarrhea, and skin problems [43-45] as PI3Kδ blockade interrupts T cells homeostasis and suppresses Treg proliferation and function [46-48]. Given the reported side-effects of Ide, DNTs were pre-treated with Ide ex vivo during expansion and washed prior to in vivo infusion.

The study disclosed herein collectively highlights that Ide treatment improves the overall fitness of DNTs to promote better engraftment and longer persistence in vivo. Further, this study was the first to investigate the persistence of DNTs and the first to show the correlation between DNT persistence and the durability of the treatment in vivo, emphasizing the importance of DNT persistence. Although this study primarily focused on investigating the effect of Ide on DNTs' function against AML, the data disclosed herein suggest Ide may also enhance therapeutic efficacy against other tumours by enhancing the overall fitness of DNTs and improving their in vivo persistence. Further, given that clinical application of both Ide and DNTs have been tested, translation of the findings from this study into clinic can be accelerated, where DNTs used in clinical trials can be expanded in the presence of Ide for enhanced efficacy and durability of response.

### Example 3: Impact of Pi3k-Delta Inhibitor on CAR-T Function

To investigate the impact of PI3Kδ inhibitor on CAR-T activity, CD4-CAR transduced DNTs expanded in the presence or absence of Idelalisib were compared.

### Materials and Methods

### Ex Vivo Isolation and Expansion of DNTs

DNT isolation and expansion were performed as described in Example 2. Briefly, healthy donor-derived PBMCs were depleted of CD4⁺ and CD8⁺ cells using CD4− and CD8− depletion cocktails (Stemcell Tech) and cultured on anti-CD3 antibody-coated plates (5 μg/ml; OKT3; Miltenyi Biotec) for 3 to 4 days in RPMI 1640 (Gibco) supplemented with 10% FBS and IL-2 (250 IU/ml; Proleukin, Novartis Pharmaceuticals). The purity of DNTs was assessed by staining cells with fluorochrome-conjugated anti-human CD3, CD4, CD8, and CD56 antibodies and flow cytometry analysis. DNTs were used 3 to 4 days after isolation for production of CAR4-DNTs. Human blood was collected from healthy donors after obtaining written informed consent and used according to a UHN Research Ethics Board-approved protocol (05-0221-T).

### CAR4 Retroviral Particle Production

The anti-CD4 retroviral CAR construct CD4 scFv-CD28-41BB-CD3Z CAR (CAR4) was generated as previously described (Vasic D et al. Allogeneic double-negative CAR-T cells inhibit tumor growth without off-tumor toxicities. Sci Immunol. 2022 Apr. 22; 7 (70): eabl3642.). Briefly, the coding sequence of an scFv derived from the antibody Ibalizumab was cloned in the MSGV1 retroviral vector, upstream of the hinge/transmembrane region, followed by CD28 and 4-1BB costimulatory domains and the cytoplasmic tail of CD3ζ. One day before transfection, Phoenix-GP cells were split and plated on poly-d-lysine-coated plates (Corning/WVR). The DNA-Lipofectamine complex used for transfection was prepared in Opti-MEM medium (Gibco) and included the RD114 envelope plasmid (Addgene), the CAR4 retroviral plasmid, and the Lipofectamine 2000 transfection reagent (Life Technologies). The complex was then added to the plates of Phoenix-GP cells, and the first and second viral harvests were collected 48 and 72 hours after transfection, respectively. CAR-CD4 retroviral particle harvests were pooled and stored at −80° C. and then thawed for CAR4 transduction as described below.

### Results

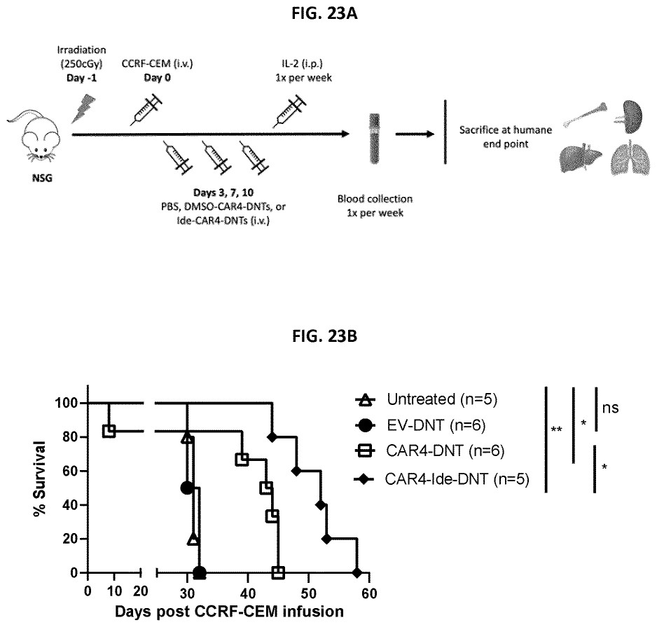

During the production of CAR4-DNTs, culture media were supplemented with Ide (CAR4-Ide-DNT). The memory status of CAR4-Ide-DNTs and CAR4-DNTs were compared. During two weeks of expansion, higher frequencies of central memory T cell (T_{CM}; CD45RA⁻CD62L⁺) and naïve/stem cell memory T cell (T_{naïve/SCM}; CD45RA⁺CD62L⁺) were observed in CAR4-Ide-DNTs. In contrast, most CAR4-DNTs were effector memory T cells (T_{EM}; CD45RA⁻CD62L⁻) with an increased frequency of terminally differentiated effector memory T cells toward later days of expansion (TEMRA; CD45RA⁺CD62L⁻) (FIG. 21).

To determine whether Ide affects the antileukemic function of CAR4-DNTs, CAR4-DNTs and CAR4-Ide-DNTs were first used in a cytotoxicity assay. After a 2-hour incubation with CCRF-CEM, CAR4-DNTs and CAR4-Ide-DNTs showed similar levels of cytotoxicity against the target (FIG. 22A). To further examine the function of CAR4-Ide-DNTs upon chronic antigen stress, CAR4-DNTs and CAR4-Ide-DNTs were used in a repeated antigen stimulation assay where fresh CCRF-CEM cells were added every 24 hours. CAR4-Ide-DNTs maintained the ability to specifically kill more than 88% CCRF-CEM cells for at least 12 rounds, whereas specific killing by CAR4-DNTs started to significantly decline after 5 rounds (FIG. 22B).

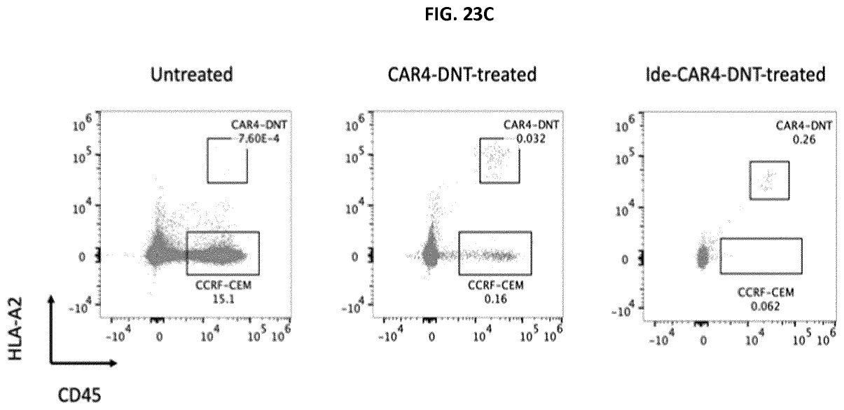

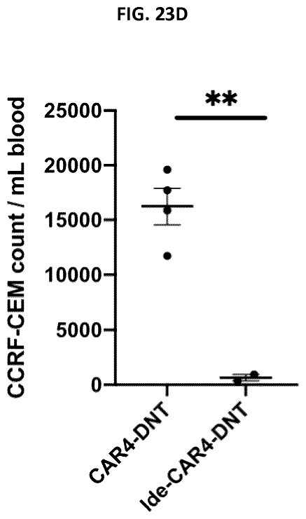

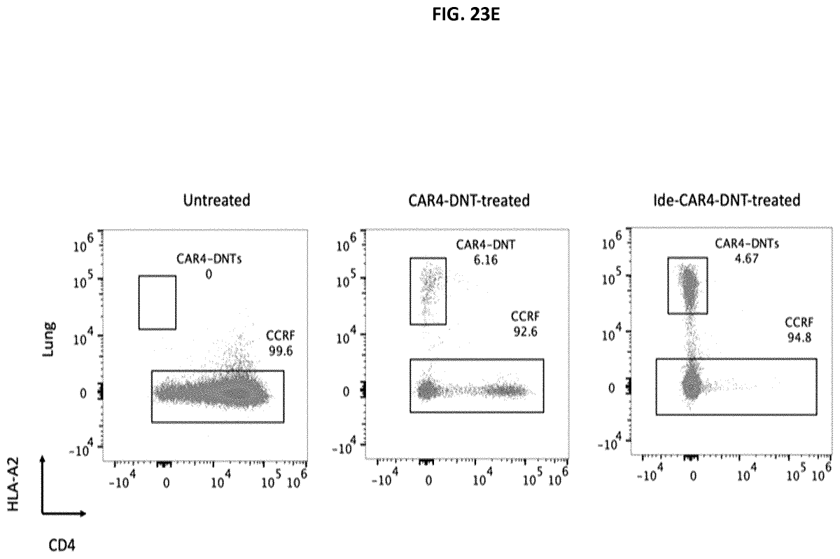

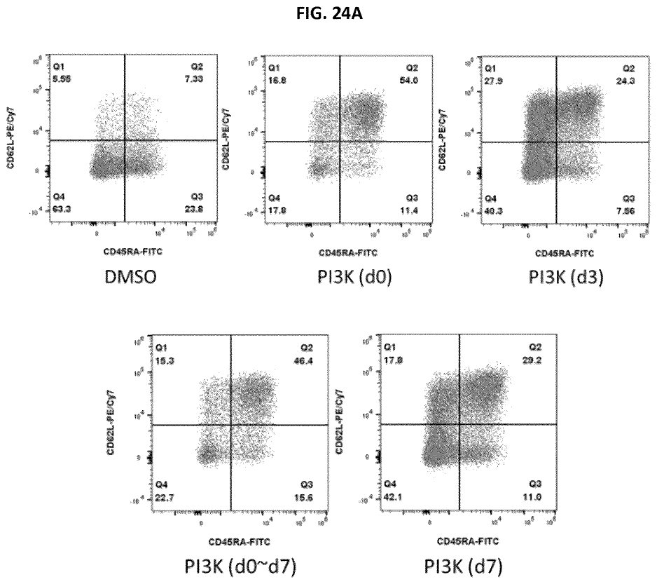

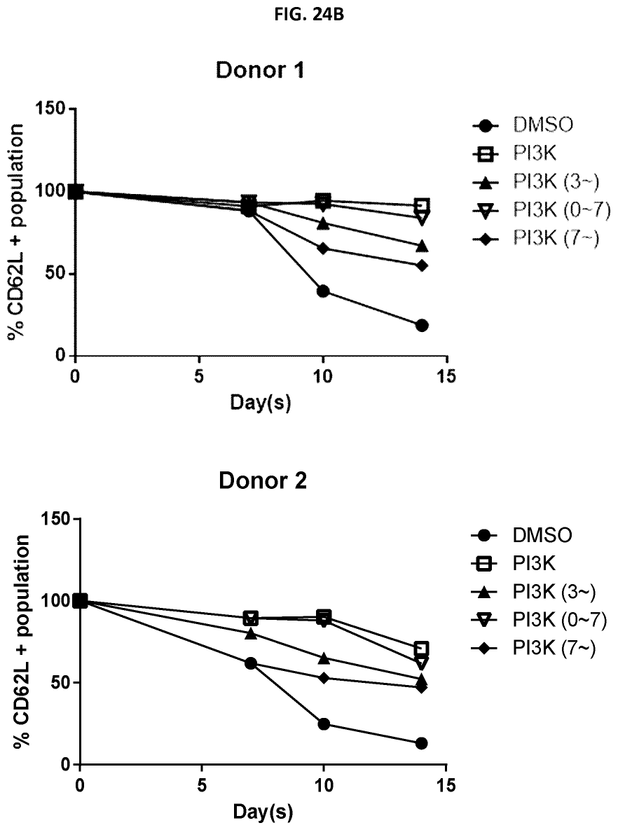

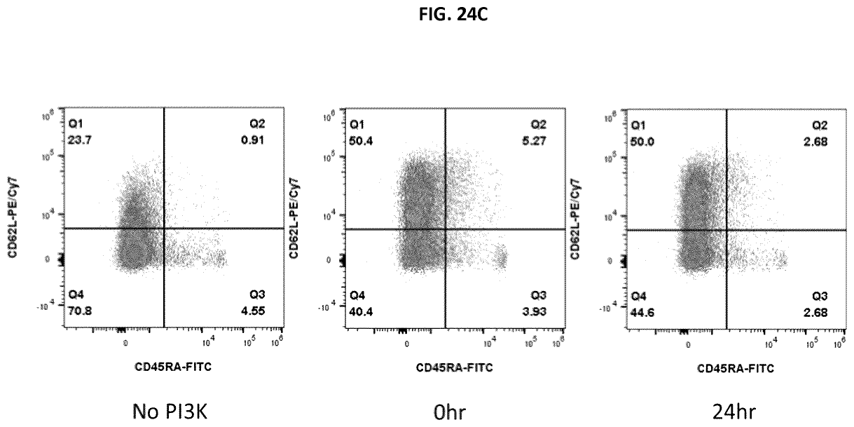

To evaluate the in vivo potency of CAR4-Ide-DNTs, CCRF-CEM-engrafted NSG mice were treated with 3 doses of PBS, CAR4-DNTs, or CAR4-Ide-DNTs (FIG. 23A). Treatment with CAR4-Ide-DNTs significantly improved the survival of mice. The median survival of mice was extended from 30 days for untreated mice and 44 days for CAR4-DNT-treated mice to 52 days for CAR4-Ide-DNT-treated mice (FIG. 23B). CAR4-Ide-DNTs significantly reduced CCRF-CEM cell counts in the peripheral blood of mice compared to both PBS and CAR4-DNTs (FIGS. 23C and 23D).

To further assess the persistence of CAR4-DNTs and the engraftment of CCRF-CEM, mouse tissues were harvested at the end point of the survival experiment. Higher levels of persisting CAR4-DNTs were detected in the CAR4-Ide-DNT-treated mice than that of CAR4-DNT mice (FIG. 23E).

### Discussion

The Ide-treated CAR-DNTs showed improved memory phenotype and were better at targeting cancer cells after multiple repeated exposures to cancer cells than untreated CAR-DNTs. Ide-treated CAR-DNTs were also better at targeting cancer cells in xenograft models. Collectively, Ide improves the anti-cancer activity of CAR-DNT similar to that seen in genetically non-modified DNTs.

While the present disclosure has been described with reference 5 to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Lee, J.-B.; Vasic, D.; Kang, H.; Fang, K. K.-L.; Zhang, L. State-of-Art of Cellular Therapy for Acute Leukemia. International Journal of Molecular Sciences 2021, 22, 4590.
2. Dohner, H.; Estey, E.; Grimwade, D.; Amadori, S.; Appelbaum, F. R.; Buchner, T.; Dombret, H.; Ebert, B. L.; Fenaux, P.; Larson, R. A.; et al. Diagnosis and management of AML in adults: 2017 ELN recommendations from an international expert panel. Blood 2017, 129, 424-447, doi:10.1182/blood-2016-08-733196.
3. Sengsayadeth, S.; Savani, B. N.; Blaise, D.; Malard, F.; Nagler, A.; Mohty, M. Reduced intensity conditioning allogeneic hematopoietic cell transplantation for adult acute myeloid leukemia in complete remission a review from the Acute Leukemia Working Party of the EBMT. Haematologica 2015, 100, 859-869, doi:10.3324/haematol.2015.123331.
4. Wang, Q. S.; Wang, Y.; Lv, H. Y.; Han, Q. W.; Fan, H.; Guo, B.; Wang, L. L.; Han, W. D. Treatment of CD33-directed chimeric antigen receptor-modified T cells in one patient with relapsed and refractory acute myeloid leukemia. Mol Ther 2015, 23, 184-191, doi:10.1038/mt.2014.164.
5. Zhang, H.; Gan, W.-T.; Hao, W.-G.; Wang, P.-F.; Li, Z.-Y.; Chang, L.-J. Successful Anti-CLL1 CAR T-Cell Therapy in Secondary Acute Myeloid Leukemia. Frontiers in Oncology 2020, 10, doi:10.3389/fonc.2020.00685.
6. Liu, F.; Cao, Y.; Pinz, K.; Ma, Y.; Wada, M.; Chen, K.; Ma, G.; Shen, J.; Tse, C.; Su, Y.; et al. First-in-Human CLL1-CD33 Compound CAR T Cell Therapy Induces Complete Remission in Patients with Refractory Acute Myeloid Leukemia: Update on Phase 1 Clinical Trial. Blood 2018, 132, 901-901.
7. Mardiana, S.; Gill, S. CAR T Cells for Acute Myeloid Leukemia: State of the Art and Future Directions. Frontiers in oncology 2020, 10, 697-697, doi:10.3389/fonc.2020.00697.
8. Boyiadzis, M.; Agha, M.; Redner, R. L.; Sehgal, A.; Im, A.; Hou, J.-Z.; Farah, R.; Dorritie, K. A.; Raptis, A.; Lim, S. H.; et al. Phase 1 clinical trial of adoptive immunotherapy using “off-the-shelf” activated natural killer cells in patients with refractory and relapsed acute myeloid leukemia. Cytotherapy 2017, 19, 1225-1232, doi:https://doi.org/10.1016/j.jcyt.2017.07.008.
9. Romee, R.; Rosario, M.; Berrien-Elliott, M. M.; Wagner, J. A.; Jewell, B. A.; Schappe, T.; Leong, J. W.; Abdel-Latif, S.; Schneider, S. E.; Willey, S.; et al. Cytokine-induced memory-like natural killer cells exhibit enhanced responses against myeloid leukemia. Sci Transl Med 2016, 8, 357ra123, doi:10.1126/scitranslmed.aaf2341.
10. Young, K. J.; Kay, L. S.; Phillips, M. J.; Zhang, L. Antitumor activity mediated by double-negative T cells. Cancer Res 2003, 63, 8014-8021.
11. Bian, Z.; Liu, J.; Xu, L. P.; Chang, Y. J.; Wang, Y.; Zhang, X. H.; Huang, X. J. Association of Epstein-Barr virus reactivation with the recovery of CD4/CD8 double-negative T lymphocytes after haploidentical hematopoietic stem cell transplantation. Bone Marrow Transplant 2017, 52, 264-269, doi:10.1038/bmt.2016.238.
12. Merims, S.; Li, X.; Joe, B.; Dokouhaki, P.; Han, M.; Childs, R. W.; Wang, Z. Y.; Gupta, V.; Minden, M. D.; Zhang, L. Anti-leukemia effect of ex vivo expanded DNT cells from AML patients: a potential novel autologous T-cell adoptive immunotherapy. Leukemia 2011, 25, 1415-1422, doi:10.1038/leu.2011.99.
13. Lee, J.; Minden, M. D.; Chen, W. C.; Streck, E.; Chen, B.; Kang, H.; Arruda, A.; Ly, D.; Der, S. D.; Kang, S.; et al. Allogeneic Human Double Negative T Cells as a Novel Immunotherapy for Acute Myeloid Leukemia and Its Underlying Mechanisms. Clinical Cancer Research 2018, 24, 370-382, doi:10.1158/1078-0432.Ccr-17-2228.
14. Yao, J.; Ly, D.; Dervovic, D.; Fang, L.; Lee, J. B.; Kang, H.; Wang, Y.-H.; Pham, N.-A.; Pan, H.; Tsao, M.-S.; et al. Human double negative T cells target lung cancer via ligand-dependent mechanisms that can be enhanced by IL-15. Journal for Immuno Therapy of Cancer 2019, 7, 17, doi:10.1186/s40425-019-0507-2.
15. Chen, J.; Hu, P.; Wu, G.; Zhou, H. Antipancreatic cancer effect of DNT cells and the underlying mechanism. Pancreatology 2019, 19, 105-113, doi:10.1016/j.pan.2018.12.006.
16. Lee, J. B.; Chen, B.; Vasic, D.; Law, A. D.; Zhang, L. Cellular immunotherapy for acute myeloid leukemia: How specific should it be? Blood Rev 2019, 35, 18-31, doi:10.1016/j.blre.2019.02.001.
17. Lee, J. B.; Kang, H.; Fang, L.; D'Souza, C.; Adeyi, O.; Zhang, L. Developing Allogeneic Double-Negative T Cells as a Novel Off-the-Shelf Adoptive Cellular Therapy for Cancer. Clin Cancer Res 2019, 25, 2241-2253, doi:10.1158/1078-0432.Ccr-18-2291.
18. Tang, B. a. L., JongBok and Cheng, Siqi and Pan, Tianzhong and Yao, Wen and Wang, Dongyao and Tu, Meijuan and Xiang, Zhiqiang and Chu, Xiandeng and Geng, Liangquan and Wang, Mingxia and Qiang, Ping and Teng, Pingping and Sun, Guangyu and Liu, Huilan and Wang, Jian and Schimmer, Aaron D. and Yang, Liming and Sun, Zimin and Zhang, Li and Zhu, Xiaoyu. A First-in-Human Phase I/IIa Study of Allogeneic Double Negative T Cell for the Treatment of Relapsed AML Post Allo-HSCT. 2021, doi:http://dx.doi.org/10.2139/ssrn.3824765.
19. Kochenderfer, J. N.; Dudley, M. E.; Feldman, S. A.; Wilson, W. H.; Spaner, D. E.; Maric, I.; Stetler-Stevenson, M.; Phan, G. Q.; Hughes, M. S.; Sherry, R. M.; et al. B-cell depletion and remissions of malignancy along with cytokine-associated toxicity in a clinical trial of anti-CD19 chimeric-antigen-receptor-transduced T cells. Blood 2012, 119, 2709-2720, doi:10.1182/blood-2011-10-384388.
20. Kochenderfer, J. N.; Dudley, M. E.; Carpenter, R. O.; Kassim, S. H.; Rose, J. J.; Telford, W. G.; Hakim, F. T.; Halverson, D. C.; Fowler, D. H.; Hardy, N. M.; et al. Donor-derived CD19-targeted T cells cause regression of malignancy persisting after allogeneic hematopoietic stem cell transplantation. Blood 2013, 122, 4129-4139, doi:10.1182/blood-2013-08-519413.
21. Porter, D. L.; Levine, B. L.; Kalos, M.; Bagg, A.; June, C. H. Chimeric Antigen Receptor-Modified T Cells in Chronic Lymphoid Leukemia. New England Journal of Medicine 2011, 365, 725-733, doi:10.1056/NEJMoa1103849.
22. Porter, D. L.; Hwang, W. T.; Frey, N. V.; Lacey, S. F.; Shaw, P. A.; Loren, A. W.; Bagg, A.; Marcucci, K. T.; Shen, A.; Gonzalez, V.; et al. Chimeric antigen receptor T cells persist and induce sustained remissions in relapsed refractory chronic lymphocytic leukemia. Sci Transl Med 2015, 7, 303ra139, doi:10.1126/scitranslmed.aac5415.
23. Turtle, C. J.; Hanafi, L. A.; Berger, C.; Gooley, T. A.; Cherian, S.; Hudecek, M.; Sommermeyer, D.; Melville, K.; Pender, B.; Budiarto, T. M.; et al. CD19 CAR-T cells of defined CD4⁺:CD8⁺ composition in adult B cell ALL patients. J Clin Invest 2016, 126, 2123-2138, doi:10.1172/jci85309.
24 Kong, W.; Lacey, S. F.; Melenhorst, J. J.; Fraietta, J. A. Biomarkers in chimeric antigen receptor T-cell therapy. Biomark Med 2018, 12, 415-418, doi:10.2217/bmm-2018-0054.
25. Mueller, K. T.; Maude, S. L.; Porter, D. L.; Frey, N.; Wood, P.; Han, X.; Waldron, E.; Chakraborty, A.; Awasthi, R.; Levine, B. L.; et al. Cellular kinetics of CTL019 in relapsed/refractory B-cell acute lymphoblastic leukemia and chronic lymphocytic leukemia. Blood 2017, 130, 2317-2325, doi:10.1182/blood-2017-06-786129.
26. Xu, S.; Ge, X.; Wang, L.; Tao, Y.; Tang, D.; Deng, X.; Yang, F.; Zhang, Q.; Qi, X.; Gong, L.; et al. Profiling pharmacokinetics of double-negative T cells and cytokines via a single intravenous administration in NSG mice. Biopharm Drug Dispos 2021, 42, 338-347, doi:10.1002/bdd.2295.
27. Waldman, A. D.; Fritz, J. M.; Lenardo, M. J. A guide to cancer immunotherapy: from T cell basic science to clinical practice. Nature Reviews Immunology 2020, 20, 651-668, doi:10.1038/s41577-020-0306-5.
28. Okkenhaug, K.; Vanhaesebroeck, B. PI3K in lymphocyte development, differentiation and activation. Nat Rev Immunol 2003, 3, 317-330, doi:10.1038/nri1056.
29. So, L.; Fruman, D. A. PI3K signalling in B- and T-lymphocytes: new developments and therapeutic advances. Biochem J 2012, 442, 465-481, doi:10.1042/bj20112092.
30. Deng, Q.; Han, G.; Puebla-Osorio, N.; Ma, M. C. J.; Strati, P.; Chasen, B.; Dai, E.; Dang, M.; Jain, N.; Yang, H.; et al. Characteristics of anti-CD19 CAR T cell infusion products associated with efficacy and toxicity in patients with large B cell lymphomas. Nat. Med. 2020, 26, 1878-1887, doi:10.1038/s41591-020-1061-7.
31. Xu, Y.; Zhang, M.; Ramos, C. A.; Durett, A.; Liu, E.; Dakhova, O.; Liu, H.; Creighton, C. J.; Gee, A. P.; Heslop, H. E.; et al. Closely related T-memory stem cells correlate with in vivo expansion of CAR.CD19-T cells and are preserved by IL-7 and IL-15. Blood 2014, 123, 3750-3759, doi:10.1182/blood-2014-01-552174.
32. Kurachi, M.; Barnitz, R. A.; Yosef, N.; Odorizzi, P. M.; Dilorio, M. A.; Lemieux, M. E.; Yates, K.; Godec, J.; Klatt, M. G.; Regev, A.; et al. The transcription factor BATF operates as an essential differentiation checkpoint in early effector CD8⁺ T cells. Nat Immunol 2014, 15, 373-383, doi:10.1038/ni.2834.
33. Pennock, N. D.; White, J. T.; Cross, E. W.; Cheney, E. E.; Tamburini, B. A.; Kedl, R. M. T cell responses: naive to memory and everything in between. Adv Physiol Educ 2013, 37, 273-283, doi:10.1152/advan.00066.2013.
34. Fraietta, J. A.; Lacey, S. F.; Orlando, E. J.; Pruteanu-Malinici, I.; Gohil, M.; Lundh, S.; Boesteanu, A. C.; Wang, Y.; O'Connor, R. S.; Hwang, W. T.; et al. Determinants of response and resistance to CD19 chimeric antigen receptor (CAR) T cell therapy of chronic lymphocytic leukemia. Nat Med 2018, 24, 563-571, doi:10.1038/s41591-018-0010-1.
35 Okkenhaug, K. Signaling by the phosphoinositide 3-kinase family in immune cells. Annu Rev Immunol 2013, 31, 675-704, doi:10.1146/annurev-immunol-032712-095946.
36. Soond, D. R.; Bjørgo, E.; Moltu, K.; Dale, V. Q.; Patton, D. T.; Torgersen, K. M.; Galleway, F.; Twomey, B.; Clark, J.; Gaston, J. S.; et al. PI3K p110delta regulates T-cell cytokine production during primary and secondary immune responses in mice and humans. Blood 2010, 115, 2203-2213, doi:10.1182/blood-2009-07-232330.
37. Yi, J. S.; Cox, M. A.; Zajac, A. J. T-cell exhaustion: characteristics, causes and conversion. Immunology 2010, 129, 474-481, doi:10.1111/j. 1365-2567.2010.03255.x.
38. Joshi, N. S.; Kaech, S. M. Effector CD8 T Cell Development: A Balancing Act between Memory Cell Potential and Terminal Differentiation. The Journal of Immunology 2008, 180, 1309-1315, doi:10.4049/jimmunol.180.3.1309.
39. Pollizzi, K. N.; Patel, C. H.; Sun, I.-H.; Oh, M.-H.; Waickman, A. T.; Wen, J.; Delgoffe, G. M.; Powell, J. D. mTORC1 and mTORC2 selectively regulate CD8⁺ T cell differentiation. The Journal of Clinical Investigation 2015, 125, 2090-2108, doi:10.1172/JCI77746.
40. Wei, J.; Long, L.; Zheng, W.; Dhungana, Y.; Lim, S. A.; Guy, C.; Wang, Y.; Wang, Y. D.; Qian, C.; Xu, B.; et al. Targeting REGNASE-1 programs long-lived effector T cells for cancer therapy. Nature 2019, 576, 471-476, doi:10.1038/s41586-019-1821-z.
41. Zheng, W.; Wei, J.; Jones, L.; Chi, H.; Geiger, T. Targeting REGNASE-1 improves efficacy of chimeric antigen receptor T cell therapy for leukemia. The Journal of Immunology 2020, 204, 239.238-239.238.
42. Weigelt, B.; Downward, J. Genomic Determinants of PI3K Pathway Inhibitor Response in Cancer. Front Oncol 2012, 2, 109, doi:10.3389/fonc.2012.00109.
43. Yang, Q.; Modi, P.; Newcomb, T.; Queva, C.; Gandhi, V. Idelalisib: First-in-Class PI3K Delta Inhibitor for the Treatment of Chronic Lymphocytic Leukemia, Small Lymphocytic Leukemia, and Follicular Lymphoma. Clin Cancer Res 2015, 21, 1537-1542, doi:10.1158/1078-0432.Ccr-14-2034.
44. Furman, R. R.; Sharman, J. P.; Coutre, S. E.; Cheson, B. D.; Pagel, J. M.; Hillmen, P.; Barrientos, J. C.; Zelenetz, A. D.; Kipps, T. J.; Flinn, I.; et al. Idelalisib and Rituximab in Relapsed Chronic Lymphocytic Leukemia. New England Journal of Medicine 2014, 370, 997-1007, doi:10.1056/NEJMoa1315226.
45 Gopal, A. K.; Kahl, B. S.; de Vos, S.; Wagner-Johnston, N. D.; Schuster, S. J.; Jurczak, W. J.; Flinn, I. W.; Flowers, C. R.; Martin, P.; Viardot, A.; et al. PI3Kδ Inhibition by Idelalisib in Patients with Relapsed Indolent Lymphoma. New England Journal of Medicine 2014, 370, 1008-1018, doi:10.1056/NEJMoa1314583.
46. Jia, Y.; Yang, Q.; Wang, Y.; Li, W.; Chen, X.; Xu, T.; Tian, Z.; Feng, M.; Zhang, L.; Tang, W.; et al. Hyperactive PI3Kδ predisposes naive T cells to activation via aerobic glycolysis programs. Cell Mol Immunol 2021, 18, 1783-1797, doi:10.1038/s41423-020-0379-x.
47. Coulter, T. I.; Chandra, A.; Bacon, C. M.; Babar, J.; Curtis, J.; Screaton, N.; Goodlad, J. R.; Farmer, G.; Steele, C. L.; Leahy, T. R.; et al. Clinical spectrum and features of activated phosphoinositide 3-kinase o syndrome: A large patient cohort study. J Allergy Clin Immunol 2017, 139, 597-606.e594, doi:10.1016/j.jaci.2016.06.021.
48. Chellappa, S.; Kushekhar, K.; Munthe, L. A.; Tjønnfjord, G. E.; Aandahl, E. M.; Okkenhaug, K.; Tasken, K. The PI3K p1100 Isoform Inhibitor Idelalisib Preferentially Inhibits Human Regulatory T Cell Function. J Immunol 2019, 202, 1397-1405, doi:10.4049/jimmunol. 1701703.

1. A method of enhancing the therapeutic efficacy of double negative (CD4⁻, CD8⁻) T cells (DNTs), comprising contacting the DNTs with a PI3Kδ inhibitor during ex vivo expansion of the DNTs to produce enhanced DNTs.

2. The method of claim 1, wherein the DNTs comprise or consist of autologous cells.

3. The method of claim 1, wherein the DNTs comprise or consist of allogenic cells.

4. The method of claim 1, wherein contacting the DNTs with the PI3Kδ inhibitor (a) increases persistence of the DNTs as compared to control DNTs that have not been contacted with the PI3Kδ inhibitor, (b) increases anti-tumor activity of the DNTs as compared to control DNTs that have not been contacted with the PI3Kδ inhibitor, (c) promotes an early memory status of the DNTs as compared to control DNTs that have not been contacted with the PI3Kδ inhibitor, and/or (d) prevents exhaustion and/or improves viability of DNTs as compared to control DNTs that have not been contacted with the PI3Kδ inhibitor.

5. (canceled)

6. (canceled)

7. (canceled)

8. The method of claim 1, wherein the PI3Kδ inhibitor is selected from the group consisting of idelalisib (Ide), PI-3065 and AZD8835.

9. The method of claim 1, wherein the PI3Kδ inhibitor is idelalisib (Ide).

10. The method of claim 9, comprising contacting the DNTs with Ide at a concentration of between about 0.1 μM and about 5.0 μM, between about 0.1 μM and about 3.0 μM, between about 0.5 μM and about 2.0 μM, or about 1.0 μM.

11. (canceled)

12. (canceled)

13. (canceled)

14. The method of claim 9, comprising contacting the DNTs with Ide for at least about 3 days, at least about 5 days, at least about 10 days, at least about 15 days or at least about 20 days and/or wherein the DNTs are contacted with Ide starting at day 0 of culture or within 7 days of expansion, wherein total expansion is from 10-20 days.

15. (canceled)

16. The method of claim 1, further comprising removing some or all of the PI3Kδ inhibitor from contact with the enhanced DNTs.

17. The method of claim 1, wherein the DNTs are CAR-DNTs, optionally CD4-CAR-DNTs or CD19-CAR-DNTs.

18. (canceled)

19. (canceled)

20. A population of enhanced DNTs produced by the method of claim 1.

21. The population of enhanced DNTs of claim 20, wherein the enhanced DNTs (a) exhibit an increased persistence as compared to control DNTs that have not been contacted with the PI3Kδ inhibitor, (b) exhibit an increased anti-tumor activity as compared to control DNTs that have not been contacted with the PI3Kδ inhibitor, (c) exhibit an early memory status as compared to control DNTs that have not been contacted with the PI3Kδ inhibitor, and/or (d) exhibit a decrease in T cell exhaustion and/or an increase in DNT viability compared to control DNTs that have not been contacted with the PI3Kδ inhibitor.

22. (canceled)

23. (canceled)

24. (canceled)

25. A pharmaceutical composition comprising the population of enhanced DNTs of claim 20 and a pharmaceutically acceptable carrier.

26. (canceled)

27. (canceled)

28. (canceled)

29. (canceled)

30. A method of treating cancer in a subject in need thereof, the method comprising administering to the subject the population of enhanced DNTs of claim 20.

31. The method of claim 30, wherein the cancer is leukemia, optionally acute myeloid leukemia.

32. The method of claim 30, wherein the enhanced DNTs are CD4-CAR-DNTs, and wherein the cancer is T cell leukemia or T cell lymphoma.

33. The method of claim 30, wherein the enhanced DNTs are produced from CD19-CAR-DNTs, and wherein the cancer is B cell leukemia or B cell lymphoma.

34. (canceled)

35. A method of reducing the growth or proliferation of a tumor, the method comprising contacting the tumor with the population of enhanced DNTs of claim 20.

36. The method of claim 35, wherein the enhanced DNTs are CD4-CAR-DNTs, and wherein the tumor is T cell leukemia or T cell lymphoma.

37. The method of claim 35, wherein the enhanced DNTs are produced from CD19-CAR-DNTs, and wherein the cancer is B cell leukemia or B cell lymphoma.
